(19) United States
(12) Patent Application Publication
(10) Pub. No.: US 2015/0126581 A1
(43) Pub. Date: May 7, 2015

(54) MicroRNAs and Uses Thereof

(71) Applicant: The University of Western Australia, Perth, Western Australia (AU)

(72) Inventors: Peter Leedman, Mt Claremont (AU); Keith Giles, Mullaloo (AU); Michael Epis, Ashfield (AU); Andrew Barker, Melville (AU)

(73) Assignee: The University of Western Australia, Perth, Western Australia (AU)

(21) Appl. No.: 14/383,704

(22) PCT Filed: Mar. 8, 2013
(86) PCT No.: PCT/AU2013/000224

§ 371 Date: Sep. 8, 2014

(30) Foreign Application Priority Data
Mar. 8, 2012 (AU) 2012900933

(51) Int. Cl. C12N 15/113; A61K 45/06; C12Q 1/68; A61K 31/7088

(52) U.S. Cl. CPC C12N 15/1137; A61K 31/7088; A61K 45/06; C12Q 1/6886; C12Q 2600/158; C12Q 2600/178
USPC 514/44 A; 435/184; 435/455; 514/44 R; 536/24.5; 435/6.12

(57) ABSTRACT

Provided herein are methods for inhibiting expression of DOHH in a cell, and for inhibiting hypusination of eIF5A in a cell, the methods comprising contacting a cell with a miRNA or a nucleic acid molecule encoding the miRNA, wherein the miRNA binds to the 3′UTR of the DOHH mRNA and wherein binding results in a reduction in DOHH expression. Also provided are methods for reducing cellular proliferation and for treating diseases associated with abnormal cellular proliferation.

### FIELD

Provided are microRNAs (miRNAs) that target the 3′ untranslated region of the deoxyhypusine hydroxylase monooxygenase (DOHH) mRNA and compositions comprising the same. Methods of making and using the miRNA, including use in diagnostic and therapeutic methods, are also provided.

### BACKGROUND

Although originally described as a protein synthesis initiation factor, eIF5A has been implicated in several biological processes, including, for example, protein translation elongation, nucleocytoplasmic transport of mRNA, mRNA stability and nonsense-mediated decay. These functions have lead to the implication of eIF5A in a variety of diseases and disorders, such that eIF5A is increasingly becoming a target for therapies for these diseases.

eIF5A is a small protein (˜18 kDa) that is highly conserved throughout eukaryotes and contains the unusual amino acid hypusine [N^{ε}-(4-amino-2-hydoxybutyl)-lysine]. Hypusine is formed by a specific posttranslational modification involving two enzymes, deoxyhypusine synthase (DHS) and deoxyhypusine hydroxylase monooxygenase (DOHH). These enzymes catalyse separate reactions to form hypusine. DHS initially forms a deoxyhypusine residue from a free lysine residue situated on inactivated eIF5A. Once the deoxyhypusine residue is formed, DOHH catalyses hydroxylation of this residue, forming hypusine.

Importantly, eIF5A is activated by the hypusination pathway, and it is the mature, activated eIF5A that is mostly involved in the biological processes that eIF5A has been associated with. Activation of eIF5A also determines its localisation within the cell, with active eIF5A being predominantly cytoplasmic and inactive eIF5A found in both the nuclear and cytoplasmic compartments.

eIF5A has been shown to have several roles in the cell. For example, eIF5A has been demonstrated to be an elongation factor, although studies in mammalian cells indicate that only a subset of mRNA species utilise this protein: perhaps as little as 5% of mRNA in quiescent cells, while it appears more vital in the expression of proteins involved in cell cycle progression in actively dividing cells. eIF5A has also been shown to specifically bind to several mRNA species, including the Nos2 transcript, HIV-derived transcripts and the transcript encoding CD83. eIF5A has also been demonstrated to bind proteins such as the HIV protein Rev and the putative tumor suppressor, exportin 4 (XPO4). These activities suggest that eIF5A could act as an mRNA shuttle between the cytoplasm and nucleus in conjunction with, for example, XPO4 or Rev.

These specific activities of eIF5A have implications in disease. For example, eIF5A has been associated with the promotion of cell proliferation, including hyperproliferation of tumor cells, and its expression has been associated with several neoplasms and cancers, such as vulvar high-grade intraepithelial neoplasia, ovarian cancer, non-small cell lung cancer, BCR-ABL-positive leukemias and hepatocellular carcinoma (see e.g. Balabanov et al. (2007) Blood 109:1701-1711; Lee et al. (2010) Int J Cancer 127(4):968-976; He et al. (2011) Int J Cancer 129(1):143-50; Cracchiolo et al. (2004) Gynecol Oncol 94:217-222). In addition, studies indicate that hypusinated eIF5A has a role in the development of diabetes mellitus, and have also implicated hypusinated eIF5A in HIV-1 replication.

Accordingly, there is need for agents that can selectively inhibit hypusination of eIF5A and therefore activation of this molecule.

### SUMMARY OF THE DISCLOSURE

The present invention relates to methods and compositions for inhibiting the expression of DOHH, and methods and compositions for treating conditions and diseases that are treatable by inhibiting the expression of DOHH, such as, for example, conditions and diseases associated with aberrant DOHH expression and/or aberrant eIF5A expression or activity. The present invention also relates to methods for the detection of cancer cells in a subject.

In one aspect, the present invention provides methods of inhibiting expression of DOHH in a cell, comprising contacting the cell with a miRNA or a nucleic acid molecule encoding the miRNA, wherein the miRNA binds to the 3′UTR of the DOHH mRNA and wherein binding results in a reduction in DOHH expression.

In a further aspect, provided are methods of inhibiting hypusination of eIF5A in a cell, comprising contacting the cell with a miRNA or a nucleic acid molecule encoding the miRNA, wherein the miRNA binds to the 3′UTR of the DOHH mRNA and wherein binding results in a reduction in DOHH expression.

In another aspect, the present invention provides methods of reducing cellular proliferation, comprising contacting a cell or tissue with a miRNA or a nucleic acid molecule encoding the miRNA, wherein the miRNA binds to the 3′UTR of the DOHH mRNA and wherein binding results in a reduction in DOHH expression.

In particular embodiments, the cell contacted by the miRNA or nucleic acid molecule encoding the miRNA in the methods described above is a cancer cell, a HIV-infected cell or an islet β cell.

Another aspect of the present invention is the provision of methods of treating a disease associated with abnormal cellular proliferation in a subject, comprising administering to the subject a miRNA or a nucleic acid molecule encoding the miRNA, wherein the miRNA binds to the 3′UTR of the DOHH mRNA and wherein binding results in a reduction in DOHH expression.

In particular embodiments, the disease associated with abnormal cellular proliferation is a cancer. In one embodiment, the cancer is selected from among biliary tract cancer; bladder cancer; breast cancer; brain cancer; glioblastoma; medulloblastoma; cervical cancer; choriocarcinoma; colon cancer; colorectal carcinoma; endometrial cancer; esophageal cancer; gastric cancer; head and neck cancer; hematological neoplasms, acute lymphocytic and myelogenous leukemia; multiple myeloma; AIDS-associated leukemia and adult T-cell leukemia lymphoma; intraepithelial neoplasms; Bowen's disease; Paget's disease; liver cancer; lung cancer; small cell lung cancer; non-small cell lung cancer; lymphoma; Hodgkin's disease; lymphocytic lymphoma; neuroblastoma; oral cancer; squamous cell carcinoma; osteosarcomas; ovarian cancer; pancreatic cancer; prostate cancer; rectal cancer; sarcoma; leiomyosarcoma; rhabdomyosarcoma; liposarcoma; fibrosarcoma; synovial sarcoma; osteosarcoma; skin cancer; melanoma; Kaposi's sarcoma; basocellular cancer; squamous cell cancer; testicular cancer; thyroid cancer; renal cancer; adenocarcinoma and Wilms tumor. In a particular example, the cancer is prostate cancer.

In a further aspect, provided are methods of reducing HIV-1 replication in a cell, comprising contacting the cell with a miRNA or a nucleic acid molecule encoding the miRNA, wherein the miRNA binds to the 3′UTR of the DOHH mRNA and wherein binding results in a reduction in DOHH expression.

Also provided are methods of treating HIV-1 in a subject, comprising administering to the subject a miRNA or a nucleic acid molecule encoding the miRNA, wherein the miRNA binds to the 3′UTR of the DOHH mRNA and wherein binding results in a reduction in DOHH expression.

In one aspect, the present invention provides methods of preventing or treating diabetes in a subject, comprising administering to the subject a miRNA or a nucleic acid molecule encoding the miRNA, wherein the miRNA binds to the 3′UTR of the DOHH mRNA and wherein binding results in a reduction in DOHH expression.

In another aspect, the present invention provides methods of sensitising cells to an anti-proliferative agent, comprising contacting the cell with a miRNA or a nucleic acid molecule encoding the miRNA, wherein the miRNA binds to the 3′UTR of the DOHH mRNA and wherein binding results in a reduction in DOHH expression.

A further aspect of the present invention is the provision of methods of enhancing the effect of an anti-proliferative agent, comprising contacting a cell with a miRNA or a nucleic acid molecule encoding the miRNA, wherein the miRNA binds to the 3′UTR of the DOHH mRNA and wherein binding results in a reduction in DOHH expression; and then contacting the cell with the anti-proliferative agent.

In some embodiments of the methods described above, the miRNA binds to one or more target sites between nucleotides 280 to 460 of the human DOHH mRNA 3′UTR set forth in SEQ ID NO:3 or corresponding nucleotides in a DOHH mRNA 3′-UTR of another species.

In one embodiment, the target site is selected from among the target sites set forth in nucleotides 286-292, 308-315, 331-338, 354-361, 377-384, 423-430 and 446-452 of the DOHH 3′-UTR set forth in SEQ ID NO:3 or corresponding nucleotides in a DOHH mRNA 3′-UTR of another species. In another embodiment, the target site is selected from among the target sites set forth in nucleotides 300-306, 323-329, 346-352, 369-375, 392-398 and 438-444 of the human DOHH mRNA 3′UTR set forth in SEQ ID NO:3 or corresponding nucleotides in a DOHH mRNA 3′-UTR of another species.

In some embodiments of the methods described above and herein, the miRNA comprises a sequence of nucleotides set forth in SEQ ID NO:8 and wherein the nucleotides set forth in SEQ ID NO:8 mediate binding of the miRNA to the 3′-UTR of the DOHH mRNA. In one example, the miRNA is hsa-miR-331-3p and comprises a sequence set forth in SEQ ID NO:4 or is a variant thereof that has at least or about 80% sequence identity to the sequence set forth in SEQ ID NO:4.

In further embodiments, the miRNA comprises a sequence of nucleotides set forth in SEQ ID NO:9 and wherein the nucleotides set forth in SEQ ID NO:9 mediate binding of the miRNA to the 3′-UTR of the DOHH mRNA. In one example, the miRNA is hsa-miR-642-5p and comprises a sequence set forth in SEQ ID NO:5 or is a variant thereof that has at least or about 80% sequence identity to the sequence set forth in SEQ ID NO:5.

In particular embodiments, the nucleic acid encoding the miRNA comprises or encodes the hsa-miR-331 precursor comprising a sequence set forth in SEQ ID NO:6 or a variant thereof that has at least or about 80% sequence identity to the sequence set forth in SEQ ID NO:6. In other embodiments, the nucleic acid encoding the miRNA comprises or encodes the hsa-miR-642a precursor comprising a sequence set forth in SEQ ID NO:7′ or a variant thereof that has at least or about 80% sequence identity to the sequence set forth in SEQ ID NO:7. In some examples, the nucleic acid encoding the miRNA is a vector.

In some aspects of the methods described above and herein, two or more miRNAs or nucleic acid molecules encoding two or more miRNAs are administered to the subject or contacted with the cell. In some embodiments, the two or more miRNAs include hsa-miR-331-3p comprising a sequence set forth in SEQ ID NO:4 or a variant thereof that has at least or about 80% sequence identity to the sequence set forth in SEQ ID NO:4; and hsa-miR-642-5p comprising a sequence set forth in SEQ ID NO:5 or a variant thereof that has at least or about 80% sequence identity to the sequence set forth in SEQ ID NO:5.

In particular embodiments, the methods described above and herein further comprise administering to the subject or contacting the cell with an additional therapeutic agent. In some examples, the therapeutic agent is selected from among an anti-cancer agent, anti-viral agent, anti-diabetic agent, immunomodulatory agent and an anti-proliferative agent. In particular embodiments, the therapeutic agent inhibits the hypusination of eIF5A. In further embodiments, the therapeutic agent inhibits DOHH or DHS activity. For one example, the therapeutic agent inhibits DOHH activity and is selected from among the group consisting of mimosine, deferiprone and ciclopirox. In other examples, the therapeutic agent inhibits DHS activity and is N1-guanyl-1,7-diaminoheptane (GC7) or guanylhydrazone CNI-1493. In still further embodiments, the therapeutic agent is a siRNA. In a particular aspect of the provided methods, the miRNA or nucleic acid molecule encoding the miRNA and the additional therapeutic agent are administered to the subject or contacted with the cell at the same time or sequentially.

Another aspect of the present invention is the provision of compositions comprising hsa-miR-331-3p comprising a sequence set forth in SEQ ID NO:4 or a variant thereof that has at least or about 80% sequence identity to the sequence set forth in SEQ ID NO:4; and hsa-miR-642-5p comprising a sequence set forth in SEQ ID NO:5 or a variant thereof that has at least or about 80% sequence identity to the sequence set forth in SEQ ID NO:5.

In a further aspect, the present invention provides methods of detecting cancer cells in a subject, comprising: measuring the level of hsa-miR-331-3p, hsa-miR-642-5p and/or DOHH mRNA or protein in a subject; and comparing the level to a reference level of hsa-miR-331-3p, hsa-miR-642-5p and/or DOHH mRNA or protein, respectively; wherein cancer cells are detected if the level of hsa-miR-331-3p and/or hsa-miR-642-5p in the subject are decreased compared to the reference level; and/or the level of DOHH mRNA or protein in the subject are increased compared to the reference level. In some embodiments of this method, only the level of hsa-miR-331-3p is measured. In other embodiments, only the level of hsa-miR-642-5p is measured. In further embodiments, the levels of hsa-miR-331-3p and hsa-miR-642-5p are measured; or the levels of hsa-miR-331-3p and DOHH mRNA or protein are measured; or the levels of hsa-miR-642-5p and DOHH mRNA or protein are measured; or the levels of hsa-miR-331-3p, hsa-miR-642-5p and DOHH mRNA or protein are measured.

In particular embodiments, the levels of hsa-miR-331-3p in the subject are decreased by at least or about 20% or more compared to the reference level. In other embodiments, the levels of hsa-miR-642-5p in the subject are decreased by at least or about 20% or more compared to the reference level. In further embodiments, the levels of DOHH mRNA or protein in the subject are increased by at least or about 20% or more compared to the reference level.

In one example of the methods of detecting cancer cells described above and herein, the ratio of DOHH mRNA or protein to hsa-miR-331-3p, and/or the ratio of DOHH mRNA or protein to hsa-miR-642-5p is determined and compared to a reference ratio of DOHH mRNA or protein to hsa-miR-331-3p and/or a reference ratio of DOHH mRNA or protein to hsa-miR-642-5p, respectively, and cancer is detected if the ratio of DOHH mRNA or protein to hsa-miR-331-3p and/or the ratio of DOHH mRNA or protein to hsa-miR-642-5p in the subject is increased compared the reference ratio. In some examples, the ratio in a subject is increased by at least or about 20% or more compared to the reference ratio. In particular embodiments, the cancer is prostate cancer.

In some embodiments of each of the methods of the present prevention described above and herein, the reduction in DOHH expression is a reduction of DOHH mRNA expression. In further embodiments, the reduction in DOHH expression is a reduction in DOHH protein expression. In still further embodiments, the reduction in DOHH expression is a reduction in DOHH mRNA expression and a reduction in DOHH protein expression.

### BRIEF DESCRIPTION OF THE DRAWINGS

Embodiments of the disclosure are described herein, by way of non-limiting example only, with reference to the following drawings.

FIG. 1 shows the interaction of hsa-miR-331-3p and hsa-miR-642-5p with the 3′-UTR of the DOHH mRNA (variant 2: Genbank Acc. No. NM_{—}031304.4). (A) Schematic representation of the DOHH mRNA containing seven hsa-miR-331-3p binding sites [(a)-(g)]and six hsa-miR-642-5p binding sites [(h)-(m)] predicted by TargetScan (Release 6.2, June 2012). (B) Sequences of the predicted DOHH 3′-UTR hsa-miR331-3p target sites. The sequences in the DOHH 3′-UTR targeted by the hsa-miR-331-3p seed sequence are shown in bold and underlined. (C) Sequences of the predicted DOHH 3′-UTR hsa-miR-642-5p target sites. The sequences in the DOHH 3′-UTR targeted by the hsa-miR-642-5p seed sequence are shown in bold and underlined.

FIG. 2 represents the results of reporter gene assays in HeLa cells demonstrating that the DOHH 3′-UTR is a target for both hsa-miR-331-3p and hsa-miR-642-5p. HeLa cells were co-transfected with a construct containing a luciferase reporter gene upstream of either the DOHH 3′-UTR or the ErbB-2 3′-UTR, and also transfected with pre-miR-331-3p, pre-miR-642-5p, or a negative control (miR-NC). Reporter gene activity was then assessed. (A) hsa-miR-331-3p targets the DOHH 3′-UTR reporter in HeLa cells as well as the ErbB-2 3′-UTR positive control. * denotes p<0.05. (B) hsa-miR-331-3p and hsa-miR-642-5p both independently and in combination target the DOHH 3-UTR in reporter gene assays. All analyses are p<0.05. * denotes significance to miR-NC, ** significant to hsa-miR-642-5p, *** significance to hsa-miR-331-3p.

FIG. 3 represents the results of experiments measuring the effect of transient expression of hsa-miR-331-3p and/or hsa-miR-642-5p on DOHH expression and cell proliferation in HeLa cells. (A) DOHH RNA and protein was observed to be targeted by transient over expression of hsa-miR-331-3p and/or hsa-miR-642-5p in HeLa cells. Levels of DOHH mRNA after hsa-miR-331-3p and/or hsa-miR-642-5p transfection were expressed as a function of miR-NC. DOHH levels were normalised to both β-actin and GAPDH using GENEX (Multi D). Immunoblots were analysed using tubulin as a loading control. (B) Transfection of hsa-miR-331-3p and/or hsa-miR-642-5p decreased the growth of HeLa cells using the xCELLigence™ System (Roche). 5000 transiently transfected HeLa cells were plated in quadruplicate in E-plates and growth was monitored over a 90 h period. Results were expressed compared to miR-NC. (C) HeLa cell proliferation was significantly inhibited by a cell cycle and DOHH inhibitor, mimosine, at 50 and 250 μM. Addition of both hsa-miR-331-3p and hsa-miR-642-5p in combination with mimosine enhanced the inhibitory effect on HeLa cell proliferation over a 72 h period. Relative inhibition miRNA combination experiments were expressed to miR-NC/0 μM mimosine.

FIG. 4 represents the results of experiments measuring expression of DOHH mRNA, hsa-miR-331-3p and hsa-miR-642-5p in prostate cancer cell lines LNCap, C4-2B, DU145 and/or 22RV1, and RWPE-1 normal prostate epithelial cells. (A) RT-qPCR analysis of DOHH mRNA expression in RWPE-1 normal prostate epithelial cells and LNCaP, C4-2B, DU145, and 22RV1 prostate cancer cells. Data are expressed relative to RWPE-1 cells, where DOHH mRNA expression was normalized to β-actin and GAPDH using GENEX software. Error bars represent S.D. *, p<0.05. (B) hsa-miR-331-3p and hsa-miR-642-5p expression in LNCaP and C4-2B prostate cancer cell lines compared with RWPE-1 cells. Error bars represent S.D.*, p<0.005.

FIG. 5 is a schematic showing the 182 base pair element targeted by hsa-miR331-3p and hsa-miR-642-5p in the 3′UTR of the DOHH mRNA variant 1 (Genbank Acc. No. NM_{—}001145165.1; SEQ ID NO:1) and variant 2 (Genbank Acc. No. NM_{—}031304.4; SEQ ID NO:2). The 182 base pair element corresponds to nucleotide positions 1632 to 1813 of the DOHH mRNA variant 1 set forth in SEQ ID NO:1 (Genbank Acc. No. NM_{—}001145165.1) and nucleotide positions 1343 to 1524 of the DOHH mRNA variant 2 set forth in SEQ ID NO:2 (Genbank Acc. No. NM_{—}031304.4).

FIG. 6 represents the results of reporter gene assays in HeLa cells demonstrating that hsa-miR-331-3p and hsa-miR-642-5p target the 182 base pair element in the DOHH 3′-UTR. (A) Three constructs were generated in the pmiREPORT vector for use in the reporter assay: the first construct contains a full length DOHH 3′-UTR operatively linked to a luciferase reporter (labelled “3′UTR”); the second construct contains a DOHH 3′-UTR in which the 182 base pair element is deleted, operatively linked to a luciferase reporter (labelled “3′UTR MINUS ELEMENT”); and the third construct contains the 182 base pair element operatively linked to a luciferase reporter (labelled “3′UTR ELEMENT”). (B) Luciferase expression from “3′UTR” and “3′UTR ELEMENT” constructs was inhibited by hsa-miR-331-3p and hsa-miR-642-5p, alone or in combination, compared to elevated expression from these constructs in the absence of the miRNAs (NC), indicating that the miRNAs target the 182 base pair element to down regulate activity. (C) Luciferase expression from “3′UTR MINUS ELEMENT” vector that lacks the 182 base pair element could not be down regulated by either hsa-miR-331-3p or hsa-miR-642-5p, alone or in combination. Effects of miRNAs on reporter vectors are relative to miR-NC where * p<0.01, ** p<0.01, *** p, 0.001.

FIG. 7 represents the results of reporter gene assays in LNCaP and C4-2B prostate cancer cells. LNCaP and C4-2B cells co-transfected with hsa-miR-331-3p and/or hsa-miR-642-5p and “3′UTR”, “3′UTR ELEMENT” or “3′UTR MINUS ELEMENT” constructs described above. Luciferase expression from “3′UTR” and “3′UTR ELEMENT” constructs was inhibited by hsa-miR-331-3p and hsa-miR-642-5p, alone or in combination, compared to elevated expression from these constructs in the absence of the miRNAs (NC), indicating that the miRNAs target the 182 base pair element to down regulate activity. Luciferase expression from “3′UTR MINUS ELEMENT” vector that lacks the 182 base pair element could not be down regulated by either hsa-miR-331-3p or hsa-miR-642-5p, alone or in combination. For each sample, firefly luciferase activity was normalized to Renilla luciferase activity. Data for each reporter construct are expressed relative to miR-NC transfected cells. Error bars represent S.D. *, p<0.05; **, p<0.0001.

FIG. 8 represents the results of experiments measuring the effect of transient expression of hsa-miR-331-3p and/or hsa-miR-642-5p on DOHH expression and cell proliferation in DU145 prostate cells. (A) RT-qPCR and Western blotting analysis of DOHH mRNA and protein expression 24 and 48 h, respectively, after transfection of DU145 cells with hsa-miR-331-3p and/or hsa-miR-642-5p or miR-NC. Levels of DOHH mRNA after hsa-miR-331-3p and/or hsa-miR-642-5p transfection were expressed as a function of miR-NC. DOHH levels were normalised to both β-actin and GAPDH. Immunoblots were analysed using tubulin as a loading control. DOHH RNA and protein was observed to be targeted by transient over expression of hsa-miR-331-3p and/or hsa-miR-642-5p in DU145 prostate cells. (B) Cell proliferation assay 5 days post transfection of DU145 cells with hsa-miR-331-3p and/or hsa-miR-642-5p, or miR-NC. Error bars represent S.D., and data are expressed relative to miR-NC/vehicle. *, p<0.05. In some instances, mimosine (75 μM) was added 48 h post transfection, and cell proliferation was analyzed 3 days later. Transfection of hsa-miR-331-3p and/or hsa-miR-642-5p decreased the growth of DU145 prostate cells. Error bars represent S.D., and data are expressed relative to miR-NC/mimosine. **, p<0.001. †, synergy according to the Bliss Additivity model. Addition of both hsa-miR-331-3p and hsa-miR-642-5p in combination with mimosine enhanced the inhibitory effect on of DU145 prostate cell proliferation.

FIG. 9 demonstrates the inverse correlation of DOHH expression with hsa-miR-331-3p and hsa-miR-642-5p expression in prostate tumours. The expression of DOHH, hsa-miR-331-3p and hsa-miR-642-5p in ten prostate cancer samples (T) and matched normal adjacent tissues (NAT) was assessed (A) hsa-miR-331-3p and DOHH expression was detected in all 10 NAT and T pairs and compared using a scatter plot. hsa-miR-331-3p is down regulated in T compared to NAT in nine of ten pairs analysed. Of these, six pairs exhibited significant DOHH over expression. (B) hsa-miR-642-5p and DOHH expression compared by scatter plot, as above. All ten pairs of NAT v T exhibited down regulation of hsa-miR-642-5p in T compared to NAT, and significantly, eight pairs displayed lower levels of hsa-miR-642-5p associated with higher levels of DOHH mRNA, in T v NAT.

FIG. 10 represents the results of RT-qPCR analysis of DOHH mRNA, hsa-miR-331-3p, and hsa-miR-642-5p expression in a cohort of five matched tumor versus NAT (N) pairs in which elevated DOHH mRNA expression was present in the tumor samples. Expression of each RNA is shown in a box plot as a ratio of tumor/normal (T/N), where >1 indicates higher expression in tumor than normal tissue, and <1 indicates lower expression in tumor than normal. Reduced miR-331-3p and miR-642-5p expression was observed in the subset of prostate cancer with elevated DOHH mRNA.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

As used herein, the singular forms “a”, “an” and “the” include plural aspects unless the context clearly dictates otherwise. Thus, for example, reference to “a miRNA” includes a single miRNA, as well as two or more miRNAs; reference to “a therapeutic agent” includes a single therapeutic agent, as well as two or more therapeutic agents; and so forth.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein, “microRNA” or “miRNA” refers to a non-coding RNA, typically between about 18 and 25 nucleotides in length that hybridizes to and regulates the expression of a coding RNA. In certain embodiments, a miRNA is the product of cleavage of a precursor (pre-miRNA), for example by the enzyme Dicer.

As used herein, “pre-miRNA” refers to a non-coding RNA having a hairpin structure, which contains a miRNA. Typically the term “pre-miRNA” refers to a precursor molecule, the processing and cleavage of which gives rise to a mature miRNA. In certain embodiments, a pre-miRNA is the product of cleavage of a pri-miR by a double-stranded RNA-specific ribonuclease.

As used herein, “inhibiting” means a reduction or suppression of a process or event, such as reduction or suppression of gene or protein expression, hypusination or cellular proliferation. Inhibition induced by a miRNA can be detected by any method known in the art that measures the process or the product of the process. For example, inhibition of expression can be assessed by measuring the amount of protein and/or mRNA produced from a gene. Inhibition of gene expression by an miRNA can result the amount of protein or mRNA produce from the gene being reduced by at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95% or more compared to the amount of protein or mRNA produced from the gene in the absence of the miRNA. Similarly, inhibition of cellular proliferation by an miRNA can result the proliferation of cells being reduced by at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95% or more compared to the cellular proliferation in the absence of the miRNA. Inhibition of hypusination of eIF5A by an miRNA can result in the amount of hypusinated eIF5A being reduced by at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95% or more compared to the amount of hypusinated eIF5A in the absence of the miRNA. The term “inhibiting” and grammatical variations thereof do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably.

As used herein, the term “activity” as it pertains to a protein, polypeptide or polynucleotide means any cellular function, action, effect or influence exerted by the protein, polypeptide or polynucleotide, either by a nucleic acid sequence or fragment thereof, or by the protein or polypeptide itself or any fragment thereof.

As used herein, the phrase “contacting the cell with a miRNA” and grammatical variations thereof includes any process in which the miRNA is brought into contact with, or ultimately introduced into, a cell. Included in such processes are any appropriate nucleic acid delivery methods, including methods that deliver naked DNA and RNA into a cell, such as transfection methods, and methods that deliver DNA and RNA into a cell via a delivery system including, for example, liposomes, polymers, microspheres, microparticles, nanoparticles, micelles, gene therapy vectors (such as viral vectors, including retroviral and adenoviral vectors), and naked DNA expression vectors. Reference to contacting the cell with an miRNA includes reference to methods that introduce the miRNA into the cell, and methods that introduce a nucleic acid molecule containing or encoding the miRNA into the cell, such that upon transcription and/or processing, the miRNA is produced within the cell.

As used herein, the phrase “administering a miRNA to a subject” and grammatical variations thereof includes administration of a mature miRNA to the subject, administration of a precursor molecule such as a pre-miRNA or pri-miR, and also includes administration of a nucleic acid encoding the miRNA or precursor to the subject, such that upon uptake of the nucleic acid into a cell in the subject, transcription and/or processing results in the expression of the miRNA in the cell.

As used herein, reference to “deoxyhypusine hydroxylase” or “DOHH” includes reference to human DOHH, encoded by the human DOHH mRNA variants set forth in SEQ ID NOs: 1 and 2, and also includes reference to DOHH from other species, including other mammalian species, such as mouse DOHH (encoded by the mouse DOHH mRNA set forth in Genbank Accession No. NM_{—}133964), rat DOHH (encoded by the rat DOHH mRNA set forth in Genbank Accession No. NM_{—}001025006), cow DOHH (encoded by the cow DOHH mRNA set forth in Genbank Accession No. NM_{—}001075886), dog DOHH (encoded by the dog DOHH mRNA set forth in Genbank Accession No. XM_{—}542178), chimpanzee DOHH (encoded by the chimpanzee DOHH mRNA set forth in Genbank Accession No. XM_{—}512268), rhesus monkey DOHH (encoded by the rhesus DOHH mRNA set forth in Genbank Accession No. NM_{—}001194157), and chicken DOHH (encoded by the chicken DOHH mRNA set forth in Genbank Accession No. NM_{—}001031413).

As used herein, reference to the “3′-UTR of the DOHH mRNA” (or grammatical variants thereof) includes reference to the 3′-UTR of the human DOHH mRNA (set forth in SEQ ID NO:3) and also includes reference to the 3′-UTR of the DOHH mRNA from other species, including other mammals, such as rhesus monkey, chimpanzee, mouse, rat, cow, dog, chimpanzee, and chicken. The 3′-UTR of the DOHH of any species can be identified using standard methods well known to those in the art. Typically, the information is provided in the Genbank entries, such as those described above and herein.

As used herein the term “expression” may refer to expression of a polypeptide or protein, or to expression of a polynucleotide or gene, depending on the context. The polynucleotide may be coding or non-coding (e.g. miRNA). Expression of a polynucleotide may be determined, for example, by measuring the production of RNA transcript levels. Expression of a protein or polypeptide may be determined, for example, by immunoassay using an antibody (ies) that bind with the polypeptide.

As used herein a “reduction in DOHH expression” with respect to DOHH expression in a cell following contact with a miRNA refers to the reduction of DOHH expression compared to the DOHH expression in a cell that has not been contacted with the miRNA. DOHH expression in a cell contacted with a miRNA can be a reduced by at least or about 10%, 15%, 20%, 25%, 30%, 35%, 40%, 45%, 50%, 55%, 60%, 65%, 70%, 75%, 80%, 85%, 90% or more compared to DOHH expression in a cell that has not been contacted with the miRNA.

As used herein, a region or sequence of a nucleic acid molecule that “mediates binding” with another nucleic acid, such as a target nucleic acid molecule, means that the nucleotides in the region or sequence participate in Watson-Crick pairing with a complementary region or sequence in the target mRNA.

As used herein, a “target site” in a mRNA is a region of nucleotides that is complementary to a region of nucleotides in an miRNA, such as the miRNA seed region. As a consequence of the complementarity, the miRNA can bind to the target site by Watson-Crick pairing.

The terms “polynucleotide” or “nucleic acid molecule” may be used herein interchangeably and refer to a single- or double-stranded polymer of deoxyribonucleotide, ribonucleotide bases or known analogues of natural nucleotides, or mixtures thereof. A “polynucleotide” or “nucleic acid molecule” comprises two or more nucleic acids including DNA, RNA, PNA, LNA or any combination thereof. The terms include reference to the specified sequence as well as to the sequence complimentary thereto, unless otherwise indicated. Polynucleotides and nucleic acid molecules may be chemically modified by a variety of means known to those skilled in the art.

As used herein, corresponding nucleotides refer to nucleotides that occur at aligned loci. Related or variant polynucleotides are aligned by any method known to those of skill in the art. Such methods typically maximize matches, and include methods such as using manual alignments and by using the numerous alignment programs available (for example, BLASTN) and others known to those of skill in the art. By aligning the sequences of polynucleotides, one skilled in the art can identify corresponding nucleotides, using identical bases as guides. For example, by aligning the sequences of DOHH transcripts, one of skill in the art can identify corresponding nucleotides using identical nucleotides as guides. These methods can be used to identify corresponding domains or regions, such as, for example, the 3′-UTR.

As used herein, nucleic acids include DNA, RNA and analogues thereof, including peptide nucleic acids (PNA), locked nucleic acids (LNA) and mixtures thereof. Nucleic acids can be single or double stranded.

The term “subject” as used herein refers to mammals and includes humans, primates, livestock animals (e.g. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

As used herein, a “subject in need thereof” refers to a subject identified as being in need of a therapy or treatment, such as treatment by administration of a miRNA. Such subjects are known to have the disease or disorder being treated, or are suspected of having or developing the disease or disorder being treated.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms.

As used herein, “amelioration” refers to the lessening of severity of at least one indicator of a condition or disease. In certain embodiments, amelioration includes a delay or slowing in the progression of one or more indicators of a condition or disease, such as a delay or slowing of cellular proliferation. The severity of indicators may be determined by subjective or objective measures which are known to those skilled in the art.

As used herein the term “associated with” when used in the context of a disease or condition “associated with” abnormal cellular proliferation means that the disease or condition may result from, result in, be characterised by, or otherwise associated with the abnormal cellular proliferation. Thus, the association between the disease or condition and the abnormal cellular proliferation may be direct or indirect and may be temporally separated.

As used herein the term “effective amount” includes within its meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

miRNAs that Target DOHH

Provided herein are microRNAs (miRNAs) that target sites within the deoxyhypusine hydroxylase monooxygenase (DOHH) mRNA 3′ untranslated region (3′-UTR). Such miRNAs down regulate DOHH mRNA expression and protein production, which in turn inhibits hypusination of eIF5A. Accordingly, the miRNAs provided herein can be used as therapeutics in diseases and conditions in which hypusinated eIF5A is involved, or wherein inhibition of hypusination resulting from reduced DOHH expression is desired. Further, the miRNAs also can be used as diagnostic markers for particular diseases.

Deoxyhypusine hydroxylase (DOHH) is the second enzyme in the two-step catalytic reaction that results in the posttranslational hypusination of eukaryotic initiation factor 5A (eIF5A), in which one specific lysine residue of the eIF5A precursor is modified to the unique amino acid hypusine. In the first step, deoxyhypusine synthase (DHS) forms a deoxyhypusine residue from a free lysine residue situated on inactivated eIF5A. Once the deoxyhypusine residue is formed, DOHH catalyses hydroxylation of the residue, forming hypusine and converting the eIF5A protein to its active mature form. This is a highly specific reaction associated with eIF5A. No other cellular protein has been identified as containing hypusine.

Human DOHH is encoded by the HLRC1 gene. Alternative splicing results in two isoforms of the human DOHH transcript: DOHH transcript variant 1 (Genbank Acc. No. NM_{—}001145165.1) is a 2050 base pair molecule having a nucleic acid sequence set forth in SEQ ID NO:1; and DOHH transcript variant 2 (Genbank Acc. No. NM_{—}031304.4) is a 1761 base pair molecule having a nucleic acid sequence set forth in SEQ ID NO:2. The two transcript variants have different 5′ UTRs but share 100% sequence identity in the coding region corresponding to nucleotides 453-1361 of SEQ ID NO:1 and nucleotides 164 to 1072 of SEQ ID NO:2 (thereby encoding the same protein) and 100% sequence identity in the 3′-UTR, which corresponds to nucleotides 1362-2050 of SEQ ID NO:1 and nucleotides 1073-1761 of SEQ ID NO:2. The nucleotide sequence of the DOHH 3′-UTR is set forth in SEQ ID NO:3.

In addition to human DOHH, the DOHH gene and transcript have been identified and sequenced in other mammals, including, but not limited to, mouse (Genbank Accession No. NM_{—}133964), rat (Genbank Accession No. NM_{—}001025006), cow (Genbank Accession No. NM_{—}001075886), dog (Genbank Accession No. XM_{—}542178), chimpanzee (Genbank Accession No. XM_{—}512268), rhesus monkey (Genbank Accession No. NM_{—}001194157), and chicken (Genbank Accession No. NM_{—}001031413).

miRNAs are a class of short, endogenous, single-stranded, non-coding RNA molecules that bind with imperfect complementarity to the 3′ untranslated regions (3′-UTRs) of target mRNAs. miRNAs are initially transcribed as long primary transcripts (pri-miRNAs or pri-miRs). These are typically processed in the nucleus by the Drosha-DGCR8 complex, producing a 60-70 nucleotide (nt) stem loop structure known as precursor miRNA (pre-miRNA). The pre-miRNA is then exported to the cytoplasm and further processed into an intermediate miRNA duplex before association with the RNA-induced silencing complex (RISC) and maturation to single stranded miRNA. Mature miRNAs interact with sites of imperfect complementarity in 3′ untranslated regions (UTRs) of target mRNAs. These targeted transcripts subsequently undergo accelerated turnover and translational down regulation.

Many miRNAs initiate and stabilise interaction with their target mRNAs by virtue of nucleotides at positions 2 to 7 or 2 to 8, known as the mRNA “seed”, which participate in Watson-Crick pairing with a complementary (or near complementary) sequence in the target mRNA. In some instances, however, there is not perfect complementarity and other nucleotides in the mRNA may also participate in base pairing with the target to stabilise the interaction. The target recognition and binding requirements of miRNAs have been well characterised, such that artificial miRNAs can be designed to target specific sequences.

miRNAs have important roles in normal cellular development and function, and altered expression of miRNAs is associated with cancer. For example, decreased expression of the let-7 miRNAs are associated with RAS oncogene overexpression and reduced survival in non-small cell lung cancer, whereas increased miR-21 expression in a range of cancers, including those of the breast, prostate, lung, colon, pancreas and stomach, is associated with reduced apoptosis, chemoresistance, and increased tumor growth.

miRNAs are thus of particular interest not only for use in diagnostics, whereby the presence or absence of a particular miRNA or miRNA profile is indicative of a disease or condition, but also as therapeutics, whereby the miRNA binds to a target region in the transcript of a target gene to reduce expression of the product of that gene.

Provided herein are miRNAs that specifically target the 3′-UTR of the DOHH transcript. Targeting of the DOHH transcript by the miRNAs down regulates DOHH transcript and protein expression. In some instances, the amount of DOHH mRNA transcript in cells is reduced by at least or about 10%, 15%, 20%, 25%, 30%, 35%, 40%, 45%, 50%, 55%, 60%, 65%, 70%, 75% or more in the presence of the miRNA, compared to levels of DOHH mRNA in the absence of the miRNA. In other examples, the amount of DOHH protein produced by cells in the presence of the miRNA is reduced by at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97% or more compared to the amount of DOHH protein produced by cells in the absence of the miRNA.

The provided miRNAs that specifically target the 3′-UTR of the DOHH transcript can bind at any position in the 3′-UTR, as long as the binding results in reduction of the amount of DOHH mRNA and/or DOHH protein in the cell compared to the amount of DOHH mRNA and/or DOHH protein in the cell in the absence of the miRNA. Such miRNAs can be identified using the methods described herein. In some instances, publically available programs and algorithms such as TargetScan (Release 6.2, June 2012) can be used to identify miRNAs with predicted binding sites in the human DOHH mRNA 3′-UTR set forth in SEQ ID NO:3 or a DOHH mRNA 3′UTR of another species. In other examples, other bioinformatics methods are used to identify miRNAs that are not necessarily identified using algorithms such as TargetScan. The identified miRNAs are then assessed for their ability to bind to the DOHH mRNA and down regulate DOHH mRNA using standard assays, such as those described in the Examples below.

In some examples, the miRNA molecules target one or more binding sites, such as 1, 2, 3, 4, 5, 6, 7, 8 or more binding sites, between approximately nucleotides (nt) 270 to 470, 271 to 452, 280 to 470, 280 to 460, 280 to 452, or 286 to 452 of the human DOHH mRNA 3′UTR set forth in SEQ ID NO:3 or corresponding nucleotides in a DOHH mRNA 3′-UTR of another species, such as corresponding nucleotides in the rhesus monkey DOHH mRNA 3′-UTR set forth in SEQ ID NO:18. For example, the miRNAs can target nt 286-292, nt 308-315, nt 331-338, nt 354-361, nt 377-384, nt 423-430 and/or nt 446-452 of the DOHH 3′-UTR set forth in SEQ ID NO:3 or corresponding nucleotides in the DOHH 3′-UTR of a different species. Accordingly, provided are miRNAs that contain a 7 nucleotide seed having the sequence 5″ CCCCUGG 3′ (SEQ ID NO:8). The presence of such a seed facilitates annealing of the miRNA to nt 286-292, nt 308-315, nt 331-338, nt 354-361, nt 377-384, nt 423-430 and/or nt 446-452 of the DOHH 3′-UTR set forth in SEQ ID NO:3 or corresponding nucleotides in a DOHH mRNA 3′-UTR of another species.

In a further example, the miRNAs provided herein bind at nt 300-306, nt 323-329, nt 346-352, nt 369-375, nt 392-398 and/or nt 438-444 of the DOHH 3′-UTR set forth in SEQ ID NO:3 or corresponding nucleotides in the DOHH 3′-UTR of a different species. Such miRNAs contain a 6 nucleotide seed having the sequence 5′-UCCCUC-3′ (SEQ ID NO:9). The presence of such a sequence facilitates annealing of the miRNA to nt 300-306, nt 323-329, nt 346-352, nt 369-375, nt 392-398 and/or nt 438-444 of the DOHH 3′-UTR set forth in SEQ ID NO:3 or corresponding nucleotides in a DOHH mRNA 3′-UTR of another species.

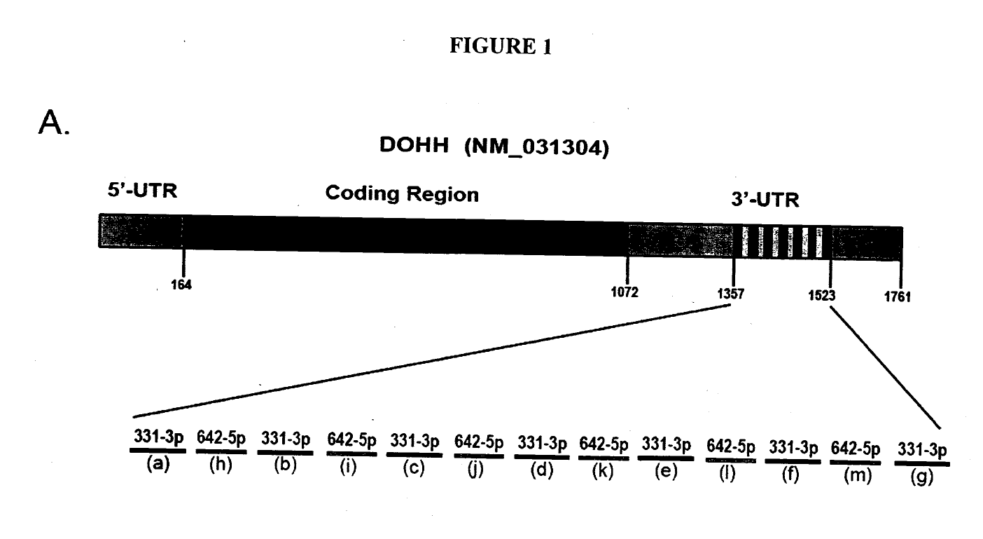

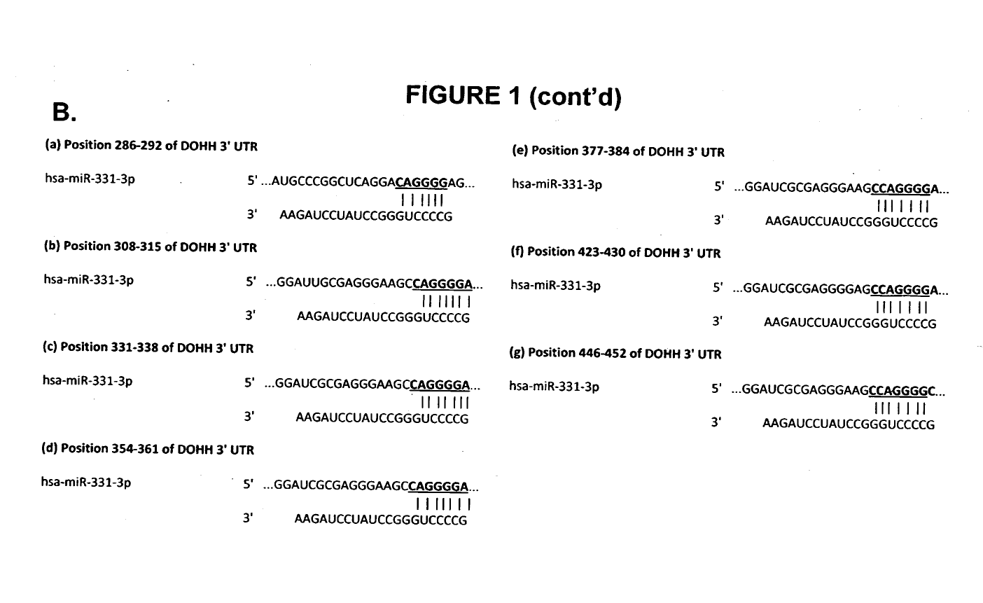

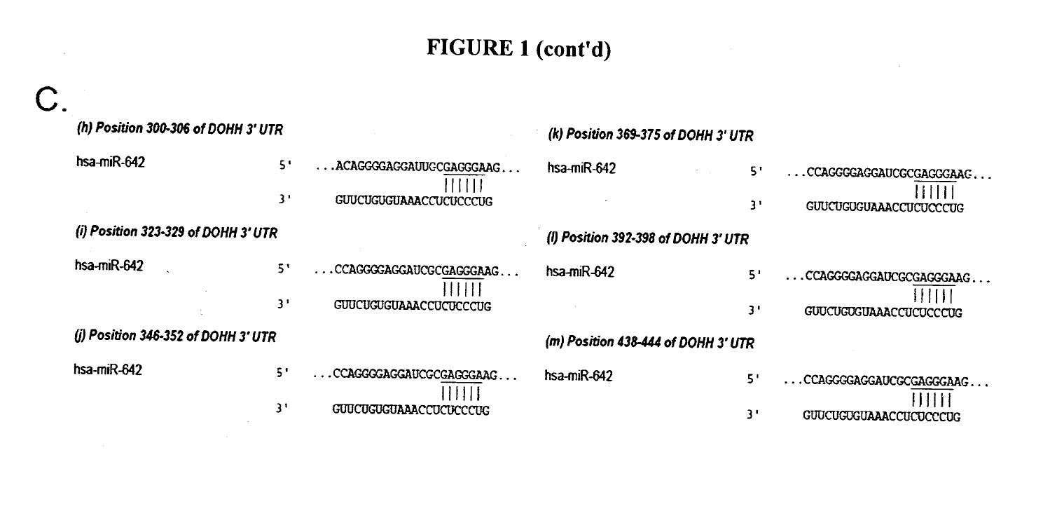

Exemplary of the miRNAs provided herein that specifically target the 3′-UTR of the DOHH transcript is human miR-331-3p (hsa-miR-331-3p) having the sequence 5′-GCCCCUGGGCCUAUCCUAGAA-3′ (SEQ ID NO:4), and variants thereof having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO:4. These variants retain the ability of human miR-331-3p to bind the 3′-UTR of the DOHH transcript and down regulate DOHH protein expression. hsa-miR-331-3p is derived from the hsa-miR-331 precursor stem loop (miRBase Acc. No. MI0000812), which has a primary sequence set forth in SEQ ID NO: 6. As described in Example 2 and shown in FIG. 1, the predicted miR-331-3p binding sites in the DOHH 3′-UTR are at nt 286-292, nt 308-315, nt 331-338, nt 354-361, nt 377-384, nt 423-430 and nt 446-452 of SEQ ID NO:3.

Also exemplary of the miRNAs provided herein that specifically target the 3′-UTR of the DOHH transcript is human miR-642-5p (hsa-miR-642-5p) having the sequence 5′-GUCCCUCUCCAAAUGUGUCUUG-3′ (SEQ ID NO:5), and variants thereof having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO:5. Such variants retain the ability of hsa-miR-642-5p to bind the 3′-UTR of the DOHH transcript and down regulate DOHH protein expression. hsa-miR-642-5p is derived from the hsa-miR-642a precursor stem loop (miRBase Acc. No. MI0003657), which has a primary sequence set forth in SEQ ID NO: 7. The predicted hsa-miR-642-5p binding sites in the DOHH 3′-UTR are at nt 300-306, nt 323-329, nt 346-352, nt 369-375, nt 392-398 and nt 438-444 of SEQ ID NO:3 (see, Example 2 and FIG. 1).

Binding of the miRNAs provided herein, including hsa-miR-331-3p or hsa-miR-642-5p or variants thereof, to the DOHH 3′-UTR in cells results in a reduction of at least or about 10%, 15%, 20%, 25%, 30%, 35%, 40%, 45%, 50%, 55%, 60%, 65%, 70%, 75% or more of DOHH transcript in the cells compared to the DOHH transcript in cells in the absence of the miRNA. Further, there is a reduction of at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97% or more of DOHH protein in the cells compared to the DOHH protein detectable in cells the absence of the miRNAs.

The miRNAs described herein that bind the 3′-UTR of DOHH and down regulate DOHH protein expression can have a sequence of nucleotides corresponding to naturally-derived miRNAs, such as a sequence corresponding to hsa-miR-331-3p or hsa-miR-642-5p set forth in SEQ ID NOs: 4 and 5, respectively, or can be a variant of a naturally-derived miRNA, such as one having at least or about 80%, 81%, 82%, 83%, 84%, 85%, 86%, 87%, 88%, 89%, 90%, 91%, 92%, 93%, 94%, 95%, 96%, 97%, 98%, or 99% identity to the sequence set forth in SEQ ID NO:4 or 5. In further examples, the miRNAs are artificial miRNAs that contain the miRNA seed set forth at nucleotides 2 to 8 of SEQ ID NO:4 and having a sequence set forth in SEQ ID NO:8, or the miRNA seed set forth at nucleotides 2 to 7 of SEQ ID NO:5 and having a sequence set forth in SEQ ID NO:9. Methods of designing artificial miRNAs that bind to a desired target mRNA based on a seed sequence are well known in the art (see e.g., de Guire et al. (2010) Nucleic Acids Research 38(13) e140). Such artificial miRNA have a seed with 100% sequence identity to the seed at nucleotides 2 to 8 of SEQ ID NO:4 or 5, but may have only 30%, 35%, 40%, 45%, 50%, 55%, 60%, 65%, 70%, 75%, 80%, 85%, 90%, 95% or more sequence identity across the entire polynucleotide set forth in SEQ ID NO:4 or 5, respectively.

Also provided are nucleic acid molecules encoding the miRNAs described herein. Included among these nucleic acid molecules are RNA molecules containing precursors of the miRNAs, such as, for example, the precursors set forth in SEQ ID NOs:6 and 7 or variants thereof, and DNA molecules encoding the miRNAs described herein, including DNA molecules encoding the precursor miRNAs described herein.

The miRNAs described herein can be produced by any method known to those of skill in the art. For example, the miRNAs and nucleic acids encoding the miRNAs can be isolated from cells or tissues, recombinantly produced, or synthesized in vitro by a variety of techniques well known in the art. The activity of the miRNAs, such as their ability to bind the 3′-UTR of the DOHH mRNA and down regulate DOHH protein expression, inhibit eIF5A hypusination, inhibit cell proliferation, inhibit HIV-1 replication and/or inhibit islet β cell destruction in diabetic inflammation, can then be tested using standard assays well known in the art.

In some examples, for the purposes of the methods described herein, the miRNAs are contained in or encoded by other nucleic acid molecules, and it is these nucleic acids that are isolated and purified for use in the described methods. For example, the miRNAs can be contained within larger RNA molecules which, when processed, produce the miRNAs described herein. In particular instances, the miRNAs are provided in larger RNA molecules such as the precursor miRNA molecules set forth in SEQ ID NOs: 6 and 7 or variants thereof having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to the precursor miRNA molecules set forth in SEQ ID NOs: 6 and 7. When processed by the cellular machinery, the miRNAs, such as hsa-miR-331-3p or hsa-miR-642-5p or variants thereof or other miRNAs described herein, are produced. In another example, the miRNAs are encoded by nucleic acid molecules, which may be contained, for example, in vectors. Thus, also provided herein are vectors that encode the miRNAs. In particular instances, the vectors encode precursor miRNAs, such as the precursor miRNA molecules set forth in SEQ ID NOs: 6 and 7 or variants thereof. The precursor miRNAs are then expressed from the vector, and the miRNAs, such as hsa-miR-331-3p or hsa-miR-642-5p or variants thereof, are produced following processing in the cell.

In some instances, the miRNAs or nucleic acids encoding the miRNA are produced synthetically using well-known methods or are isolated from cells or tissues. Typically, the miRNAs or nucleic acid molecules containing or encoding the miRNAs are obtained using genetic engineering techniques to produce a recombinant nucleic acid molecule, which can then be isolated or purified by techniques well known to one of ordinary skill in the art. In these recombinant methods, nucleic acid encoding the miRNA is cloned into an appropriate expression vector. It is well within the skill of a skilled artisan to design DNA that encodes a miRNA provided herein.

Any suitable host/vector system can be used to express one or more of the miRNAs described herein. It is well with the skill of those in the art to select an appropriate system based on, for example, whether the miRNA or nucleic acid molecule encoding the miRNA is being isolated and purified for subsequent use, and/or whether the miRNA will be expressed in vivo following administration to a subject.

In particular examples, the miRNAs described herein (including precursor miRNAs) are encoded by vectors for expression of the miRNA in vivo following administration of the vector to a subject. The choice of vector, including the particular regulatory elements contained in the vector for expression of heterologous nucleic acid, can be influenced by the cell type to which the vector is being targeted, and such selection is well within the level of skill of the skilled artisan. For example, the nucleic acid encoding the miRNA can be under the control of a tissue- or cell-specific promoter, such that the miRNA is only expressed in that particular tissue or cell type. Tissue- or cell-specific promoters are well known in the art. For example, the prostate-specific antigen (PSA) promoter and the probasin promoter, including the composite probasin promoter, ARR(2)PB, can be used to target prostate tissue. In other examples, the nucleic acid encoding the miRNA is under the control of a ubiquitous promoter.

In further examples, the nucleic acid encoding the miRNA is cloned into a viral vector, including, but not limited to, retroviral, adenoviral, lentiviral and adeno-associated viral vectors. Although viral vectors can be replication incompetent or replication competent, for subsequent use in therapeutic applications, typically replication incompetent vectors are selected.

The activity of the miRNAs can be assessed using in vitro assays and animal models well known to those skilled in the art. The miRNAs also can be assessed in human clinical trials under appropriate supervision.

The ability of the provided miRNAs to bind the 3′-UTR of the DOHH mRNA can be assessed using standard methods in the art, including, but not limited to, northern blot assays or real time PCR (qRT-PCR). The ability of the miRNAs to down regulate DOHH mRNA and/or protein expression also can be readily assessed using in vitro assays such as the reporter assays described in Example 2. Other methods that can be used include, but are not limited to, Western blot and immunostaining techniques, wherein cells that express DOHH are exposed to the miRNA and the downregulation of DOHH expression in the cells is detected using an anti-DOHH antibody by Western blot of the cell lysate or immunostaining of the cells.

The ability of the miRNAs to inhibit hypusination and activation of eIF5A also can be assessed. For example, levels of hypsusinated eIF5A in cells following exposure to the described miRNAs can be assessed using an antibody such as NIH-353 that preferentially binds to hypsusinated eIF5A (Hoqu et al. (2009) Retrovirol 6:90; Cracchiolo et al. (2004) Gynecol Oncol 94:217-222). Levels of hypusination also can be assessed using isoelectric focusing (IEF) analysis (Li et al. (2010) PLoS ONE 5(4): e9942).

The effect of the miRNAs on cellular proliferation can be assessed using standard techniques, including commercially available assays such as the xCELLigence™ System (Roche) described in Example 4, below. Other assays to assess cellular proliferation include, but are not limited to, assays that measure tritiated thymidine (3H-thymidine) or bromodeoxyuridine (BrdU) uptake, utilise propidium iodide staining, or assays that measure tetrazolium salt reduction resulting from proliferation. Exemplary tetrazolium salts that can be used in the assays include, for example, MTT (3-(4,5-dimethyl)thiazol-2-yl)-2,5-diphenyltetrazolium bromide), XTT (sodium 3′-[1-phenylamino)-carbonyl]-3,4-tetrazolium]-bis(4-methoxy-6-nitro)benzene-sulfonic acid hydrate, MTS (3-(4,5-dimethylthiazol-2-yl)-5-(3-carboxymethoxyphenyl)-2-(4-sulfophenyl)-2H-tetrazolium) and WST-1 (4-[3-(4-iodophenyl)-2-(4-nitrophenyl)-2H-5-tetrazolio]-1,3-benzene disulfonate). Any cell of interest can be used such assays. Typically, the assays utilise cancer or tumor cells or cell lines well known to those skilled in the art. For example, PC-3 or DU 145, two human prostate carcinoma cell lines established from a bone and brain metastasis respectively, can be used in the assays.

Small animal models also can be used to assess the anti-proliferative affects of the described miRNAs. Small animal cancer models are routinely used in the art and include, for example, immunocompromised animals, such as Nude, Rag1, Scid and Nod Scid mice, that have been transplanted with cancer cells, such as human cancer cells. For example, a human prostate cancer model that is established by transplanting Nod Scid mice subcutaneously or orthotopically with prostate cells, such as PC-3 and DU 145 cells, can be used to assess the ability of the miRNAs to inhibit proliferation of prostate cancer cells (Bastide et al. (2002) Prostate Cancer and Prostatic Diseases 5:311-315).

The provided miRNAs also can be assessed for their ability to inhibit HIV-1 replication. HIV-1 replication assays are well known in the art and can be used to compare HIV-1 replication in the presence and absence of the provided miRNAs (for review of HIV-1 replication assays, see e.g. McMahon et al. (2009) Curr Opin Infect Dis. 22(6):574-82).

The anti-diabetic activities of the described miRNAs also can be assessed using methods well known in the art. For example, the ability of the miRNAs to reduce islet cell loss and hyperglycaemia can be assessed using a mouse model of islet inflammation, such as the multiple low-dose streptozotocin model (Maier et al. (2010) J Clin Invest 120:2156-2170).

Methods of Using the miRNAs as Therapeutic Agents

Provided herein are methods of using miRNAs that specifically target the 3′-UTR of the DOHH transcript and down regulate DOHH expression. The miRNAs can be used in methods of inhibiting expression of DOHH protein in a cell, and methods of inhibiting activation of eIF5A in a cell. In such methods, the cell is contacted, either in vitro or in vivo, with a sufficient amount of the miRNA to result in a reduction in DOHH protein expression or eIF5A activation, respectively, compared to cells that have not been contacted with the miRNA. For example, following contact of the cell with the miRNA, the amount of DOHH produced, or the amount of eIF5A activation, can be reduced by at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95% or more compared to eIF5A activation of cells not contacted with the miRNA. Any cell type can be targeted in such methods. In particular examples, the cells include, but are not limited to, cancer cells, virus-infected cells, such as HIV-infected cells, and islet β cells.

In further examples, the miRNAs are used in methods of reducing cellular proliferation. In such methods, cells or tissue are contacted either in vitro or in vivo with a sufficient amount of the miRNA to result in a reduction in DOHH protein expression and a reduction in cellular proliferation compared to cells or tissue that have not been contacted with the miRNA. Cellular proliferation can be reduced by, for example, at least or about 10%, 15%, 20%, 25%, 30%, 35%, 40%, 45%, 50%, 55%, 60%, 65%, 70%, 75%, 80%, 85%, 90%, 91%, 92%, 93%, 94%, 95% or more compared to the proliferation of cells that have not been contacted with the miRNA. Although any proliferating cell can be contacted with the miRNAs in such methods, typically, the cell is a cancer cell.

As a result of their ability to inhibit DOHH protein expression and subsequently inhibit eIF5A activation, the miRNAs described herein also can be used in the prevention, treatment or amelioration of any disease or condition in which activated eIF5A is a pathogenic factor. Administration of the miRNAs, or nucleic acid molecules or vectors containing or encoding the miRNAs, to a subject in need thereof can result in reduced expression of DOHH in target cells, in turn resulting in reduced hypusination and activation of eIF5A. Thus, in subjects with a disease or disorder or subjects suspected of having a disease or disorders (i.e. subjects exhibiting one or more clinical indicators of a disease or disorder) in which activated eIF5A is a pathogenic factor, the development or progression of the disease or disorder can be reduced by administration of a miRNA provided herein.

The miRNAs described herein that specifically target the 3′-UTR of the DOHH transcript are useful in the treatment, prevention and/or amelioration of diseases associated with abnormal proliferation. As demonstrated in the Examples below, the miRNAs described herein, including hsa-miR-331-3p and hsa-miR-642-5p and variants thereof, down regulate expression of DOHH in cells and reduce cellular proliferation. Accordingly, the miRNAs can be administered to a subject in need thereof to prevent the development or reduce the progression of diseases associated with abnormal cellular proliferation.

Diseases associated with abnormal cell proliferation include, for example, diseases associated with pathological cellular hyperproliferation, such as malignant diseases, as well as benign tumors, pre-cancers, neoplasms, hyperplasias, polyps, warts and other growths, and autoimmune diseases characterized by hyperproliferating clones of autoreactive lymphocytes. For example, the miRNAs can be used to treat vulvar high-grade intraepithelial neoplasia, which is associated with hypusinated eIF5A. Diseases associated with abnormal proliferation also include particular degenerative disorders, Alzheimer's disease, Parkinson's disease, amyotrophic lateral sclerosis (ALS), retinitis pigmentosa, and osteoporosis.

Exemplary of diseases associated with abnormal cellular proliferation are cancers, including in solid phase tumors and malignancies, soft tissue sarcomas, and blood cell malignancies. Administration of the miRNAs, alone or in combination with other miRNAs or other therapeutic agents or other therapies, can reduce proliferation of the cancer cells, reduce tumor size and/or slow the rate of tumor growth or metastasis.

Cancers that can be treated by administration of the miRNAs described herein include, but are not limited to, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias, BCR-ABL-positive leukemias and adult T-cell leukemia lymphoma; intraepithelial neoplasms including Bowen's disease and Paget's disease; liver cancer, including human hepatocellular carcinona (HCC); lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. In particular examples, the miRNAs are used in the treatment of subjects with prostate cancer, ovarian cancer, non-small cell lung cancer, BCR-ABL-positive leukemias and/or hepatocellular carcinoma.

The miRNAs described herein that down regulate DOHH expression can also be administered to subjects infected with HIV-1 to reduce viral load. HIV-1 replication requires the viral Rev protein, which facilitates nucleocytoplasmic transport of incompletely spliced and unspliced viral mRNAs by shuttling between the nucleus and the cytoplasm. Studies show that eIF5A is a cofactor of Rev, thus implicating eIF5A in the metabolism of HIV mRNAs (Bevec et al. (1996) Science 271:1858-1860). Further studies demonstrate that blocking eIF5A activation by inhibiting DHS (the first enzyme in the hypusination reaction) results in inhibition of viral replication (Hauber et al. (2005) J Clin Invest 115:76-85). Similarly, preventing hypusination of eIF5A using drugs that inhibit DOHH activity blocked HIV-1 replication in vitro (Hogue et al. (2009) Retrovirology 6:90. Accordingly, the miRNAs described herein, including hsa-miR-331-3p and hsa-miR-642-5p and variants thereof, can be used to reduce HIV-1 replication in subjects infected with HIV-1.

In a further example, the miRNAs described herein can be used in the treatment of subjects with diabetes mellitus. Diabetes type 1 and diabetes type 2 are the result of distinctly different pathogenic mechanisms, namely the loss of insulin-producing β cells and the resistance of β cells to insulin, respectively. Nonetheless, there are some common mechanisms in the development of the diseases. For example, in both conditions, pro-inflammatory cytokines destroy the β cells in a process that involves the activation of the Nos2 gene, which encodes nitric oxide synthase (iNos). Nitric oxide synthase is responsible for the production of nitric oxide, which results in reduced ATP production and cellular necrosis.

Studies have indicated that activated eIF5A specifically binds to the Nos2 transcript and is involved in the transportation of the transcript across the nuclear membrane. Conversely, inhibition of hypusination adversely affects the interaction between eIF5A and Nos2, resulting in Nos2 mRNA being partially trapped within the nucleus and unable to be translated to iNOS (Maier et al. (2010) Discovery Med 10(50):18-23). In addition, treatment of obese diabetic mice with the DHS inhibitor GC7 resulted in improved glucose tolerance, increased insulin release, and enhanced β cell mass (Robbins et al. (2010) J Biol Chem 285(51):39943-39952). Thus, the miRNAs described herein, including hsa-miR-331-3p and hsa-miR-642-5p and variants thereof, can be used to prevent or reduce progression of diabetes.

Formulations and methods of delivery of nucleic acid molecules are well known in the art and can readily be used and adapted by a skilled person in the art for the methods herein. In some instances, the miRNAs described herein that target the 3′-UTR of DOHH, such as hsa-miR-331-3p or hsa-miR-642-5p or variants thereof, or any combination thereof, can be formulated for administration to a subject as pharmaceutical compositions containing naked miRNA molecules. In other instances, the miRNAs or nucleic acid encoding the miRNAs are formulated as pharmaceutical compositions for administration using a delivery system such as liposomes, polymers, microspheres, gene therapy vectors, and naked DNA vectors.

Generally, the compositions containing the miRNAs or nucleic acid encoding the miRNAs are prepared in view of approval from a regulatory agency or otherwise prepared in accordance with generally recognized pharmacopeia for use in animals and in humans. The compositions can include carriers such as a diluent, excipient, or vehicle. Such pharmaceutical carriers can be sterile liquids, such as water and oils. Saline solutions and aqueous dextrose and glycerol solutions also can be employed as liquid carriers, particularly for injectable solutions. Compositions can contain, in addition to the miRNAs or nucleic acid encoding the miRNAs, a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A pharmaceutical composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

The compositions can be formulated for administration by any route. The most appropriate route of administration can be determined by a person of skill in the art, taking into account the particular disease or condition being treated. Exemplary routes of administration include, for example, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

In circumstances where it is required that appropriate concentrations of the agent are delivered directly to the site in the body to be treated, administration may be regional rather than systemic. Regional administration provides the capability of delivering very high local concentrations of the agent to the required site and thus is suitable for achieving the desired therapeutic or preventative effect whilst avoiding exposure of other organs of the body to the agent and thereby potentially reducing side effects.

The formulations can be administered to a subject in therapeutically effective amounts (e.g., amounts which prevent or reduce progression of a disease or condition) to provide therapy for a disease or condition. The precise amount or dose of the miRNA or nucleic acid encoding the miRNA that is administered to the subject depends on several factors, including, but not limited to, the activity of the miRNA, the use of other therapeutic agents, the route of administration, the number of dosages administered, and other considerations, such as the weight, age and general state of the subject. Particular dosages can be empirically determined or extrapolated from, for example, studies in animal models or previous studies in humans.

The pharmaceutical compositions containing the miRNA or nucleic acid molecule encoding the miRNA can be administered by any method and route understood to be suitable by a skilled artisan, including, but not limited to, intramuscular, intradermal, transdermal, parenteral, intravenous, subcutaneous, intranasal, oral, intraperitoneal or topical administration, as well as by any combination of any two or more thereof, formulated in a manner suitable for each route of administration.

Compositions of the present disclosure may include one or more pharmaceutically acceptable carriers or diluents. Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The provided miRNAs and nucleic acid molecules encoding the miRNAs can be administered to a subject alone or in combination with each other. When administered in combination, the miRNAs or nucleic acid molecules encoding the miRNAs can be in the same or different compositions and can be administered at the same time or sequentially to one another. Accordingly, provided herein are compositions, such as pharmaceutical compositions, that contain two or more miRNAs or nucleic acid molecules encoding the miRNAs, such as hsa-331-3p or a variant thereof and hsa-miR-642-5p or a variant thereof.

Further, the miRNAs or nucleic acid molecules encoding the miRNAs can be administered in combination with one or more other therapeutic agents. In some examples, the miRNAs or nucleic acid molecules encoding the miRNAs are formulated with the one or more other therapeutic agents in the same composition. In other examples, they are formulated with the one or more other therapeutic agent in different compositions, and administered at the same time or sequentially to one another. Further, the miRNAs or nucleic acid molecules encoding the miRNAs can be administered to a subject in combination with other therapies, including, but not limited to, surgery or radiotherapy.

In particular examples, the miRNAs or nucleic acid molecules encoding the miRNAs are administered in combination with an anti-proliferative agent, such as an agent that targets eIF5A or an anti-cancer agent. Prior administration of the miRNAs or nucleic acid molecules encoding the miRNAs can sensitize the cells to the effects of the subsequently administered anti-proliferative agent, such that the overall anti-proliferative effect is enhanced compared to administration of the anti-proliferative agent alone.

In some examples, the miRNAs or nucleic acid molecules encoding the miRNAs provided herein, alone or in combination with other miRNAs or nucleic acid molecules encoding the miRNAs, are administered to a subject in combination with another agent that targets, either directly or indirectly, eIF5A. These include, but are not limited to, siRNAs and miRNAs that inhibit eIF5A, DHS and/or DOHH protein expression, and drugs or other agents, such as antibodies and fragments thereof, which inhibit DOHH or DHS activity, thereby inhibiting hypusination of eIF5A. Exemplary drugs that inhibit DOHH activity and that can be used in combination treatments with DOHH-targeting miRNAs in the methods provided herein include, for example, mimosine, deferiprone and ciclopirox. Exemplary agents that inhibit DHS activity and that can be used in combination treatments with DOHH-targeting miRNAs in the methods provided herein include, for example, spermidine-related compound N1-guanyl-1,7-diaminoheptane (GC7) and the guanylhydrazone CNI-1493 (AXD455).

The miRNAs also can be administered in combination with one or more anti-cancer agents, including, but not limited to, anti-cancer antibodies and fragments thereof, targeted anti-cancer therapeutics such as erlotinib and lapatinib, radionuclides and/or chemotherapeutics, such as abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine (BCNU), cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6th edition (Feb. 15, 2001), Lippincott Williams & Wilkins Publishers.

In further examples, the miRNAs are administered to a subject infected with HIV in combination with anti-retroviral therapy (ART). Exemplary anti-retroviral agents that can be used in combination with the miRNAs include entry or fusion inhibitors, such as maraviroc and enfuvirtide; CCR5 receptor antagonists; nucleoside and nucleotide reverse transcriptase inhibitors (NRTI); non-nucleoside reverse transcriptase inhibitors (NNRTI); protease inhibitors (PIs); and integrase inhibitors.

Other combination treatments that are contemplated herein are those for use in diabetic or pre-diabetic subjects. In such treatments, the provided miRNAs are administered to a diabetic or pre-diabetic subjection in combination with one or more other anti-diabetic agents. Exemplary anti-diabetic agents include, but are not limited to, insulins, including insulin analogs; biguanides, including metformin, phenformin and buformin; thiazolidinediones (TZDs), including rosiglitazone, pioglitazone and troglitazone; sulfonylureas, including tolbutamide, acetohexamide, tolazamide, chlorpropamide, glipizide, glyburide, glimepiride, and gliclazide; meglitinides, including repaglinide and nateglinide; alpha-glucosidase inhibitors, including miglitol and acarbose; peptide analogs, including GLP peptide analogs; and DPP-4 inhibitors.

Use of the miRNAs in Methods of Diagnosing or Detecting Cancer

Provided are methods of diagnosing or detecting a disease or condition based on the expression of one or more of the miRNAs described herein. Exemplary of the diseases or conditions that can be detected or diagnosed using the methods are diseases and conditions associated with abnormal cellular proliferation, including, for example, neoplasms, cancers and tumors. Exemplary of the cancers that can be detected or diagnosed using the methods provided herein is prostate cancer.

The methods provided herein are based on the observation that each of hsa-miR-331-3p and hsa-miR-642-5p are down regulated in some cancer cells compared to healthy tissue. The levels of hsa-miR-331-3p or hsa-miR-642-5p are reduced in these cancer cells by at least or about 20%, 25%, 30%, 35%, 40%, 45%, 50%, 55%, 60%, 65%, 70%, 75%, 80%, 85%, 90% or more compared to the levels of hsa-miR-331-3p or hsa-miR-642-5p in healthy tissue. Conversely, DOHH mRNA and protein is up regulated in these cancer cells compared to healthy tissue (see Example 6, below). The levels of DOHH mRNA and/or protein are increased in these cancer cells by at least or about 20%, 30%, 40%, 50%, 60%, 70%, 80%, 90%, 100%, 120%, 140%, 160%, 180%, 200%, 250%, 300%, 350%, 400%, 500% or more compared to the levels of DOHH mRNA and/or protein in healthy tissue.

Accordingly, the provided methods include measuring the levels of one or more of hsa-miR-331-3p, hsa-miR-642-5p and DOHH (mRNA or protein) in a sample from a subject. The levels of hsa-miR-331-3p, hsa-miR-642-5p and/or DOHH detected in the sample of the subject is then compared to a reference level of hsa-miR-331-3p, hsa-miR-642-5p and/or DOHH, wherein the reference level is determined using healthy tissue or body fluid from a healthy individual. Levels of hsa-miR-331-3p and/or hsa-miR-642-5p that are decreased, such as by at least or about 20%, 25%, 30%, 35%, 40%, 45%, 50%, 55%, 60%, 65%, 70%, 75%, 80%, 85%, 90% or more, in the sample from the subject compared to the reference levels are indicative of cancer. An increase in levels of DOHH mRNA by at least or about 20%, 30%, 40%, 50%, 60%, 70%, 80%, 90%, 100%, 120%, 140%, 160%, 180%, 200%, 250%, 300%, 350%, 400%, 500% or more, in the sample from the subject compared to the reference levels also are indicative of cancer.

Reference levels of hsa-miR-331-3p, hsa-miR-642-5p, DOHH mRNA and DOHH protein in samples can be pre-determined using samples from a cohort of healthy subjects to obtain an accurate median or mean. In other examples, the reference levels are determined using healthy tissue from subjects with a disease or disorder. Further, reference levels can be determined for various samples, such as various cell and tissue types and various body fluids. For the most accurate detection, the reference level used for comparison is the reference level obtained from the same type of sample (e.g. the same type of tissue or cell) as taken from the subject for assessment in the provided methods. The reference levels also can be matched by age, sex or other factor.

In some examples, only the levels of hsa-miR-331-3p in a sample are measured in the provided methods. In other examples, only the levels of hsa-miR-642-5p are measured. In further instances, only the levels of DOHH mRNA or protein are measured. In particular embodiments, a combination of markers are assessed. For example, the methods provided herein can include measurement of hsa-miR-331-3p and hsa-miR-642-5p levels; hsa-miR-331-3p and DOHH mRNA and/or protein; hsa-miR-642-5p and DOHH mRNA and/or protein; or hsa-miR-331-3p, hsa-miR-642-5p and DOHH mRNA and/or protein.

In methods in which a miRNA, such as hsa-miR-331-3p and/or hsa-miR-642-5p, and DOHH mRNA or protein is measured, a ratio of DOHH levels to miRNA levels or miRNA levels to DOHH levels can be determined and compared to the corresponding reference ratio determined using a sample from healthy tissue and/or healthy subjects. For example, the ratio of DOHH mRNA or protein to hsa-miR-331-3p in a sample from a subject can be determined and compared to the reference ratio. An increased ratio of DOHH to miRNA (hsa-miR-331-3p or hsa-miR-642-5p) is indicative of cancer. The ratio can be increased by at least or about 20%, 30%, 40%, 50%, 60%, 70%, 80%, 90%, 100%, 120%, 140%, 160%, 180%, 200%, 250%, 300%, 350%, 400%, 500% or more compared to the reference ratio. Likewise, a decreased ratio of miRNA (hsa-miR 331-3p or hsa-miR-642-5p) to DOHH is also indicative of cancer. The ratio can be decreased by at least or about 20%, 25%, 30%, 35%, 40%, 45%, 50%, 55%, 60%, 65%, 70%, 75%, 80%, 85%, 90% or more compared to the reference ratio.

Any method of measuring RNA or protein levels in sample can be used in the methods herein. Exemplary methods for detection of RNA, such as miRNA or mRNA include, for example, real time quantitative RT-PCR, a technique well known to those of skill in the art. Exemplary methods for detecting protein levels include, for example, immunological techniques such as ELISA, and mass spectrometry methods.

Any appropriate sample can be assessed using the methods provided herein, and it is well within the skill of those in the art to determine what type of sample is most appropriate for detecting a particular cancer. Exemplary samples include, but are not limited to, cell and tissue samples, such as from a biopsy, and biological fluids, including, but not limited to, blood, serum, urine, synovial fluid, lymph, saliva, mucus and cerebrospinal fluid.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

### Example 1

### Identification of DOHH as a Predicted Target of Hsa-miR-331 and Hsa-miR-642-5p

The publicly available target predication program TargetScan (Release 6.2: June 2012; targetscan.org; Lewis et al. (2005) Cell 120(1), 15-20) was used to identify potential targets of hsa-miR-331-3p. This analysis predicted that the DOHH mRNA 3′-UTR contains seven putative hsa-miR-331-3p binding sites at nucleotides (nt) 286-292 (labelled [a]), nt 308-315 (labelled [b]), nt 331-338 (labelled [c]), nt 354-361 (labelled [d]), nt 377-384 (labelled [e]), nt 423-430 (labelled [f]) and nt 446-452 (labelled [g]) (FIG. 1A., B.). Target site [a] has a context score of 34% on TargetScan, which is might suggest a poor quality target site, whilst the sites [b] through [g] have higher context scores ranging from 62% to 91% and have a higher degree of conservation in the seed region (Table 1). An analysis of the sequences of the hsa-miR-331-3p sites in human, mouse, rat and dog DOHH indicated that there was no sequence conservation. However, there was some conservation observed in the 3′-UTR of the DOHH mRNA from rhesus macaque, with two hsa-miR-331-3p sites observed in this region.

TABLE 1

| miR-331-3p | Seed Binding | Context score | Conserved (C) or |
|---|---|---|---|
| target site | Location (nt) | (%) | Poorly conserved (P) |
| (a) | 286-292 | 29 | P |
| (b) | 308-315 | 92 | P |
| (c) | 331-338 | 91 | C |
| (d) | 354-361 | 91 | C |
| (e) | 377-384 | 92 | C |
| (f) | 423-430 | 91 | P |
| (g) | 446-452 | 64 | P |

A bioinformatics approach (TargetScan Release 6.2, June 2012) further predicted that another miRNA, hsa-miR-642-5p, targeted the DOHH mRNA 3′-UTR, with multiple predicted putative binding sites (×6) at nt 300 306 (labelled [h]), nt 323-329 (labelled [i]), nt 346-352 (labelled [j]), nt 369-375 (labelled [k]), nt 392-398 (labelled [I]) and nt 438-444 (labelled [m]) (FIG. 1A., C.). TargetScan context scores for each of the hsa-miR-642-5p sites were significantly lower than all but one of the miR-331-3p sites listed (Table 2). Interestingly, the predicted target sites for hsa-miR-331-3p and hsa-miR-642-5p are arranged alternatively adjacent to comprise a 160 bp 3′-UTR element. This element appears to have an unusually high concentration of seed motifs specific to these two miRNAs and exists in its entirety in humans only, and partially in rhesus macaques (Data not shown).

TABLE 2

| miR-642-5p | Seed Binding | Context score | Conserved (C) or |
|---|---|---|---|
| target site | Location (nt) | (%) | Poorly conserved (P) |
| (h) | 300-306 | 39 | P |
| (i) | 323-329 | 32 | P |
| (j) | 346-352 | 32 | P |
| (k) | 369-375 | 33 | P |
| (l) | 392-398 | 35 | P |
| (m) | 438-444 | 35 | P |

### Example 2

### Reporter Gene Assays to Confirm DOHH Targeting

Reporter gene assays were performed to confirm that the 3′-UTR of DOHH was a target for hsa-miR-331-3p and hsa-miR-642-5p, as predicted using TargetScan.

### DNA Constructs

Synthetic miRNA precursor molecules corresponding to human miR-331-3p (Pre-miR miRNA Precursor Product ID: PM10881), human miR-642-5p (Pre-miR miRNA Precursor Product ID: PM11477) and a negative control miRNA (miR-NC; Pre-miR miRNA Precursor Negative Control #1, Product ID: AM 17110) were obtained from Ambion.

The dual luciferase (firefly and Renilla) reporter miRNA target clone for the DOHH 3′-UTR (catalogue no. HmiT020194-MT01) and the negative control vector (catalogue no. CmiT000001-MT01), both based on the PETZ-MT01 vector containing a SV40 promoter upstream of the hLuc gene, and a CMV reporter upstream of the hRLuc gene, were sourced from Genecopoeia™ (Rockville, USA). For miRNA target clones, the DOHH 3′-UTR was inserted downstream of the SV40 promoter/firefly luciferase reporter gene. Firefly luciferase was normalised to Renilla′ luciferase for expression and transfection efficiency.

Transfection of miRNA Precursors and Luciferase Assays

HeLa cells were seeded in 12-well plates and co-transfected using Lipofectamine 2000 (Invitrogen) with 100 ng of either the negative control (empty), DOHH 3′-UTR or ERBB-2 3′-UTR reporter construct (Genecopoeia™) and 1-2 nM final concentration of pre-miR-331-3p, pre-miR-642-5p, a combination of pre-miR-331-3p and pre-miR-642-5p, or pre-miR-NC per well. Lysates were assayed for Firefly and Renilla luciferase activities 24 h after transfection using the Dual Luciferase Reporter Assay System (Promega) and a Fluostar OPTIMA microplate reader (BMG Labtech), and firefly luciferase activities normalised to Renilla luciferase activities to obtain relative reporter gene activity.

### Results

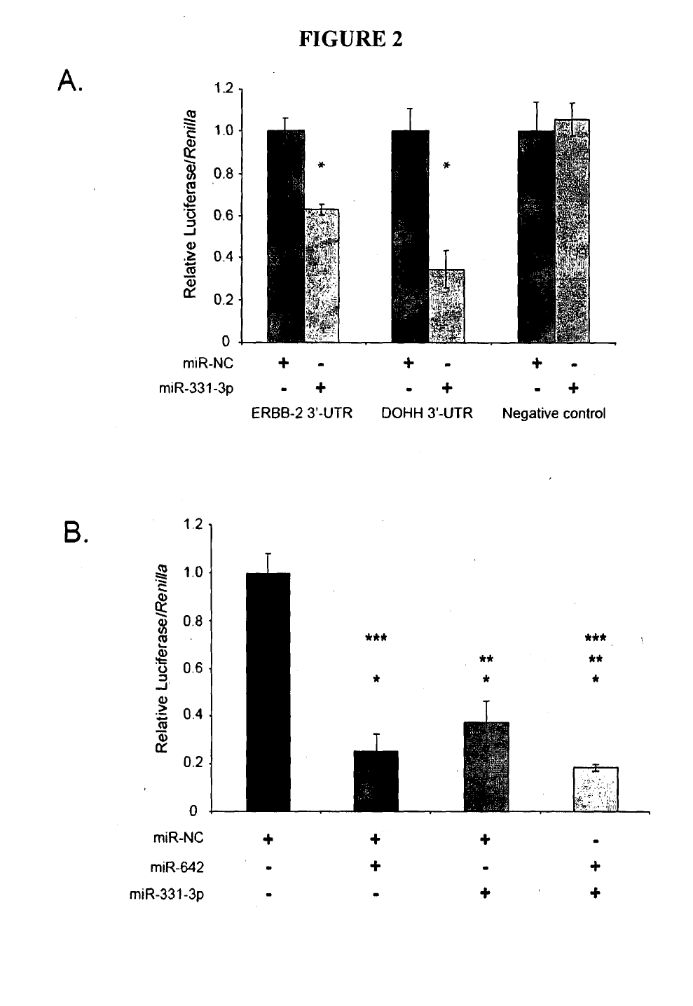

In cells co-transfected with hsa-miR-331-3p precursor and a DOHH 3′-UTR reporter, there was a significant reduction in reporter activity, compared to cells with miR-NC precursor (FIG. 2A). Similarly, in cells co transfected with miR-331-3p precursor and the ERBB-2 3′-UTR reporter (a positive control for miR-331-3p), there was also a significant reduction in reporter activity, as published previously (Epis et al. (2009) J Biol Chem 284(37), 24696-24704). HeLA cells transfected with the negative control reporter vector and either miR-NC or hsa-miR-331-3p displayed no significant change in reporter activity (FIG. 2A), indicating that the DOHH 3′-UTR was a specific target for miR-hsa-331-3p.

It was observed that hsa-miR-642-5p had an increased effect on the DOHH 3′UTR compared to the effect of hsa-miR-331-3p, while the combination of both miRNAs appeared to have a more pronounced effect again on the DOHH 3′-UTR (FIG. 2B).

Using independence criterion for Bliss Independence (which states that the expected effect for a combination of two or three agents can be calculated from the single agent effects), it was calculated that the effect of combining the miRNAs, when compared to that of the miRNAs alone, produced an additive and not synergistic effect on the repression of the DOHH 3′-UTR reporter gene.

### Example 3

### Effect of Hsa-miR-331-3p and Hsa-miR-642-5p in HeLa Cells

The effect of transient transfection of HeLa cells with hsa-miR-331-3p and hsa-miR-642-5p on DOHH expression and cell proliferation was investigated.

### A. Regulation of DOHH Expression in HeLa Cells

Having established that the DOHH 3′-UTR is a target for hsa-miR-331-3p and hsa-miR-642-5p in reporter gene assays, the effect of hsa-miR-331-3p and/or hsa-miR-642-5p over-expression on DOHH RNA and protein expression in HeLa cells was investigated. Briefly, HeLa cancer cells were seeded into 6 well plates or 10 cm² dishes and transfected using Lipofectamine 2000 (Invitrogen) and precursor miRNA molecules at a final concentration of 30 nM. Cells were harvested 24 hours post transfection for total RNA and 3 days post transfection for protein. The effect of hsa-miR-331-3p and hsa-miR-642-5p over-expression on DOHH RNA and protein production was analysed by RT-PCR and Western blot as described below.

### Reverse Transcription and Polymerase Chain Reaction (PCR)

Total RNA was extracted from transfected HeLa cells using Qiazol reagent (Qiagen). To assess DOHH, GAPDH and B-actin mRNA by qRT-PCR, 125 ng of total RNA was reverse transcribed to cDNA using the Quantitect Reverse transcription Kit (Qiagen). Real-time PCR was then performed using a Corbett 6000 Rotor-Gene™ instrument (Corbett Research) using Sensimix™ PLUS SYBR (Quantace) and primers from Primer Blast (NCBI; www.ncbi.nlm.nih.gov/tools/primerblast/index.cgi?LINK_LOC=BlastHome) (DOHH-91 and -191 primers), Primer Bank (http://pga.mgh.harvard.edu/primerbank/;25) (GAPDH and B-actin) or using validated primer assays from Qiagen (Qiagen™ Quantitect Primer Assays) for ActB, Cat# QT01680476; GAPDH, Cat# QT01192646; ERBB-2, Cat# QT00060746; DOHH, Cat# QT00235536). Sequences for primer sets (from Primer Blast and Primer Bank) were as follows;

DOHH-F91,
(SEQ ID NO: 10)
5′-GGC CGA GGG GTC CTG AGT CT-3′
DOHH R91,
(SEQ ID NO: 11)
5′-TGG GTC CCG GCC TTC CAC AA-3′
DOHH-F191,
(SEQ ID NO: 12)
5′-CAG GGC GCT GAA TCG GCA CA -3′
DOHH-R191,
(SEQ ID NO: 13)
5′TGC ATG AGG GAG GCC CGG AA 3′
GAPDH-F,
(SEQ ID NO: 14)
5′-ATG GGG AAG GTG AAG GTC G-3′
GAPDH-R,
(SEQ ID NO: 15)
5′-GGG GTC ATT GAT GGC AAC AAT A-3′
B-actin-F,
(SEQ ID NO: 16)
5′-GCC AAC ACA GTG CTG TCT GG-3′
B-actin-R,
(SEQ ID NO: 17)
5′TAC TCC TGC TTG CTG ATC CA-3′

Expression of DOHH mRNA relative to GAPDH and B-actin mRNA was determined using the 2-ΔΔCt method and statistical analysis of qRT PCR data was performed using GenEx software (MultiD).

### Western Blotting

Cytoplasmic protein extracts of the transfected HeLa cells were prepared as described previously (Giles et al. (2003) J Biol Chem 278(5), 2937-2946), resolved on NuPAGE 4-12% Bis Tris gels or NuPAGE 10% Bis Tris gels (Invitrogen) and transferred to PVDF membranes (Roche). Membranes were blocked in 5% skim milk/TBST and probed with anti-tubulin rat polyclonal antibody (1:1,000, Abcam ab6161 100) as a loading control and anti-DOHH (C 19) goat polyclonal antibody (1:1000, Santa Cruz Biotechnology sc-55157). Secondary horseradish peroxidise linked anti-rat-IgG (ab6734-1; Abcam) and anti-sheep/goat-IgG (AB324P; Chemicon) antibodies were used at 1:10000 prior to detection with ECL Plus detection reagent and ECL-Hyperfilm (GE Healthcare).

### Results

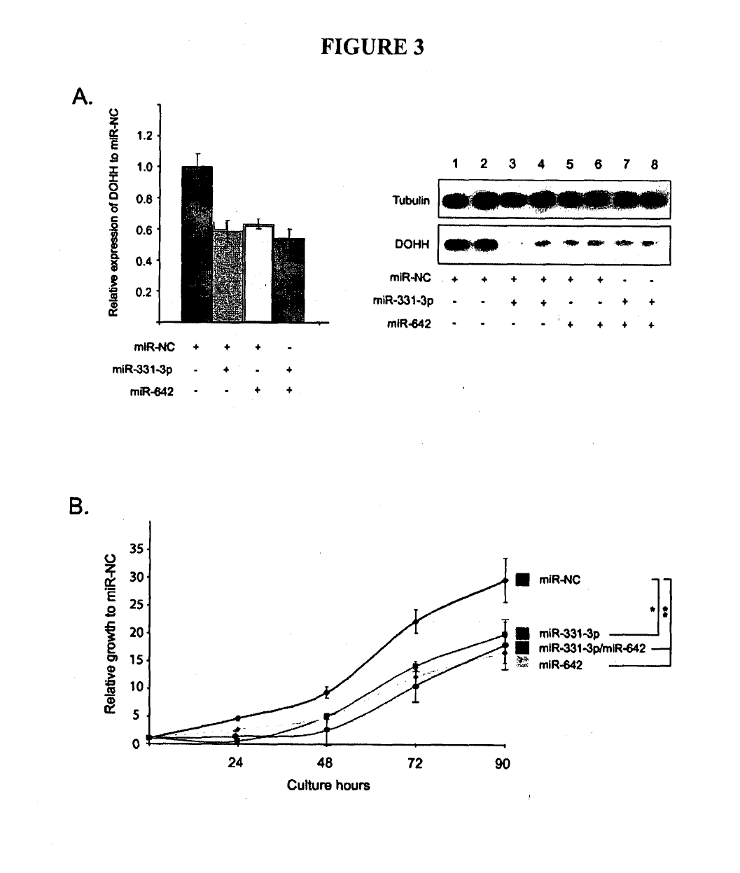

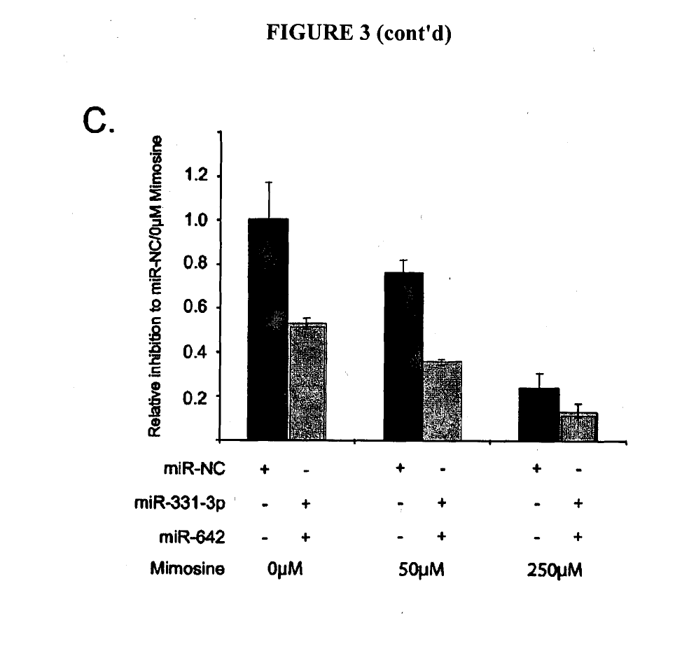

Endogenous DOHH mRNA was repressed by both hsa-miR-331-3p and hsa-miR-642-5p in HeLa cells (FIG. 3A). The data indicate that the effect of these miRNAs on endogenous DOHH protein expression was predominantly a consequence of its mRNA degradation (FIG. 3A Lanes 1-8). It was observed that hsa-miR-642-5p had a similar down regulating effect on DOHH to that of miR 331-3p and that a combination of the miRNAs, at the same total miRNA concentration (30 nM), resulted in a similar result to that achieved using the reporter gene constructs. There were unchanged cytoplasmic levels of eIF5A in HeLa cells transiently over expressing hsa-miR-331-3p, hsa-miR-642-5p or a combination (data not shown: assessed by immunoblotting with a total anti-eIF5A antibody (Abcam®; Anti-eIF5A antibody [EP526Y] (ab32443); Rabbit monoclonal antibody).

These data indicate that hsa-miR-331-3p and/or hsa-miR-642-5p over expression effects endogenous DOHH expression in the HeLa cancer cell line, and confirms the specificity of DOHH as a target of hsa-miR-331-3p and hsa-miR-642-5p. Furthermore, the combination of hsa-miR-331-3p and hsa-miR-642-5p has an additive, not synergistic effect on DOHH expression, which was previously seen in reporter gene assays. The altered expression of DOHH by hsa-miR-331-3p and hsa-miR-642-5p did not have an effect on global cytoplasmic eIF5A expression (data not shown).

B. Effect of Transient Transfection of Hsa-miR-331-3p and/or Hsa-miR-642-5p on Growth of HeLa Cancer Cells

The effect of transient miRNA over-expression in HeLa cells on cell proliferation was investigated using the xCELLigence™ System (Roche). Briefly, HeLa cells were grown and transfected with the miRNA negative control, hsa-miR-331-3p and/or hsa-miR-642-5p precursors as described above. Transfected HeLa cells were then trypsinised and counted using the Countess™ Automated cell counter and 5000 transiently transfected HeLa cells were plated in quadruplicate into 16 well E-plates. The cells monitored over a 90 hour period in real time according to the manufacturer's instructions.

The data indicate that transient transfection of both hsa-miR-331-3p and hsa-miR-642-5p, alone or in combination, inhibit the proliferation of HeLa cells to similar degrees compared to cells that were transiently transfected with the miRNA negative control (FIG. 3B).

C. Effect of Transient Transfection of Lisa-miR-331-3p and/or Hsa-miR-642-5p on Mimosine Action in HeLa Cancer Cells

The effect of the combination of hsa-miR-331-3p and hsa-miR-642-5p on the transient effects of the DOHH inhibitor mimosine in HeLa cells was assessed. Initially, the xCELLigence™ System was used but growth curves could not be established using mimosine that reflected the observed changes in cell viability (data not shown). It was observed that increasing concentrations of the drug changed the morphology of HeLa cells from a spindle like epithelial morphology to a large, round and flattened morphology, which rendered the xCELLigence™ System (which measures electrical impedance as a surrogate of cell proliferation) inappropriate for the measurement of mimosine's effects on cell proliferation. This was attributed to the ability of mimosine to arrest cell in GI and increase cell size by up to 25%.

Endpoint growth assays were then performed using the CellTiter 96® AQueous One Solution Cell Proliferation System (MTS) (Promega, USA) to determine effects of miRNAs and mimosine on cell proliferation. HeLa cells were transiently transfected with miRNA negative control or hsa-miR-331-3p/hsa-miR-642-5p as described above, and 5000 transiently transfected HeLa cells were plated in 96 well plates. Plates were incubated in a 37° C. incubator (5% CO₂) for 2 days and then exposed for a further 3 days to mimosine (0, 50 or 250 μM). Media was then replaced with a MTS substrate/growth media mixture (180 μL of growth media, 20 μL MTS reagent) and left for 30 min at 37° C. Absorbance was measured using a Fluostar Optima Instrument (Walker scientific) at 450 nm.

A significant block of cell proliferation was observed when the cells were exposed to mimosine alone (50 and 250 μM) at 3 d (Day 5 post miRNA transfection). This block of cell proliferation was further enhanced by the addition of the combination of hsa-miR 331 3p and hsa-miR-642-5p (FIG. 3C). This result was attributed to the down regulation of DOHH, initially via hsa-miR-331-3p and hsa-miR-642-5p and further by mimosine.

### Example 4

### DOHH mRNA, Hsa-miR-331-3p and Hsa-miR-642-5p Expression in Prostate Cancer Cells

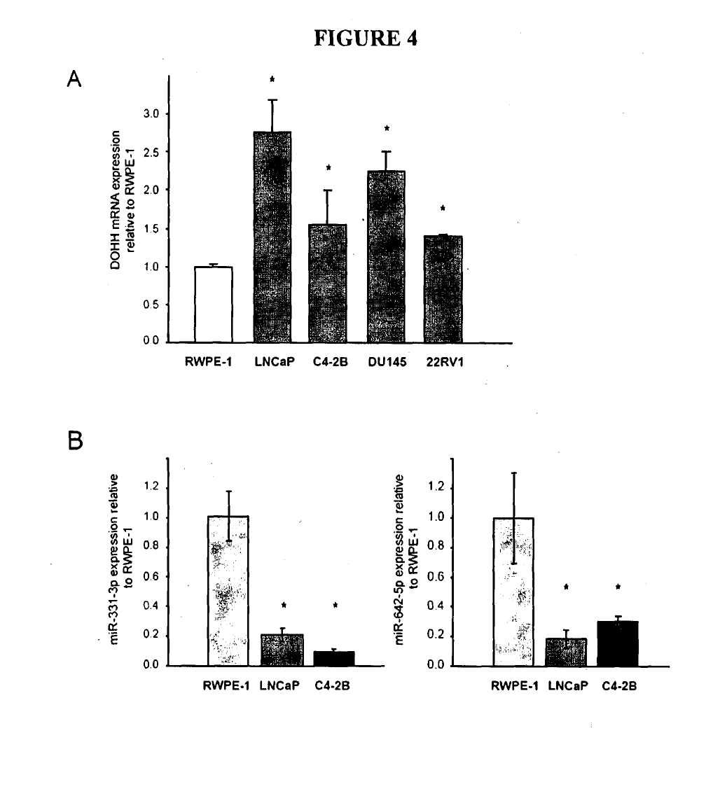

The expression of DOHH mRNA, hsa-miR-331-3p and hsa-miR-642-5p in prostate cancer cell lines LNCap, C4-2B, DU145 and/or 22RV1 was assessed as described above in Example 3, and compared to expression in RWPE-1 normal prostate epithelial cells (all cells available from ATCC). DOHH mRNA was found to be significantly up-regulated in all prostate cancer cell lines (FIG. 4A) compared to normal RWPE-1 cells. Conversely, miR-331-3p and miR-642-5p expression was significantly down regulated in LNCaP and C4-2B prostate cancer cells compared with RWPE-1 cells (FIG. 4B). This inverse relationship between DOHH mRNA and miR-331-3p and miR-642-5p expression in prostate cancer cell lines suggests the potential for these miRNAs to regulate DOHH expression in the prostate.

### Example 5

### Determination of the Region in the DOHH 3′UTR Targeted by Hsa-miR-331-3p and Hsa-miR-642-5p

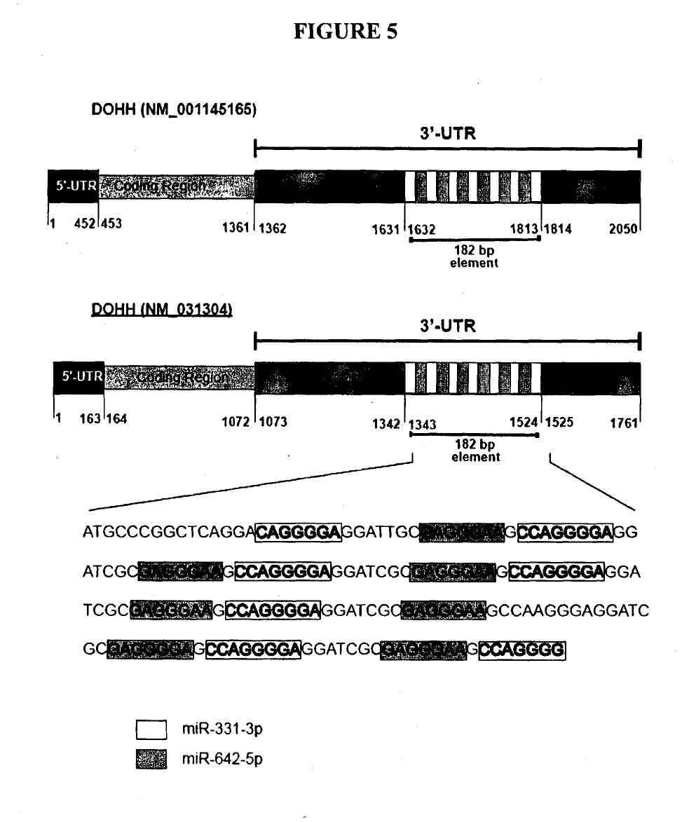

To confirm that the regions in the 3′UTR of DOHH that were predicted to be targeted by hsa-miR-331-3p and hsa-miR-642-5p (see Example 1 and FIG. 1) were indeed targeted, reporter constructs with and without a 182 nucleotide element containing the predicted targeted regions were generated and used in reporter gene assays. As shown in FIG. 5, the 182 nucleotide element corresponds to nucleotide positions 1632 to 1813 of the DOHH mRNA variant 1 set forth in SEQ ID NO:1 (Genbank Acc. No. NM_{—}001145165.1), nucleotide positions 1343 to 1524 of the DOHH mRNA variant 2 set forth in SEQ ID NO:2 (Genbank Acc. No. NM_{—}031304.4) and nucleotide positions 271-452 of the 3′UTR of DOHH set forth in SEQ ID NO:3.

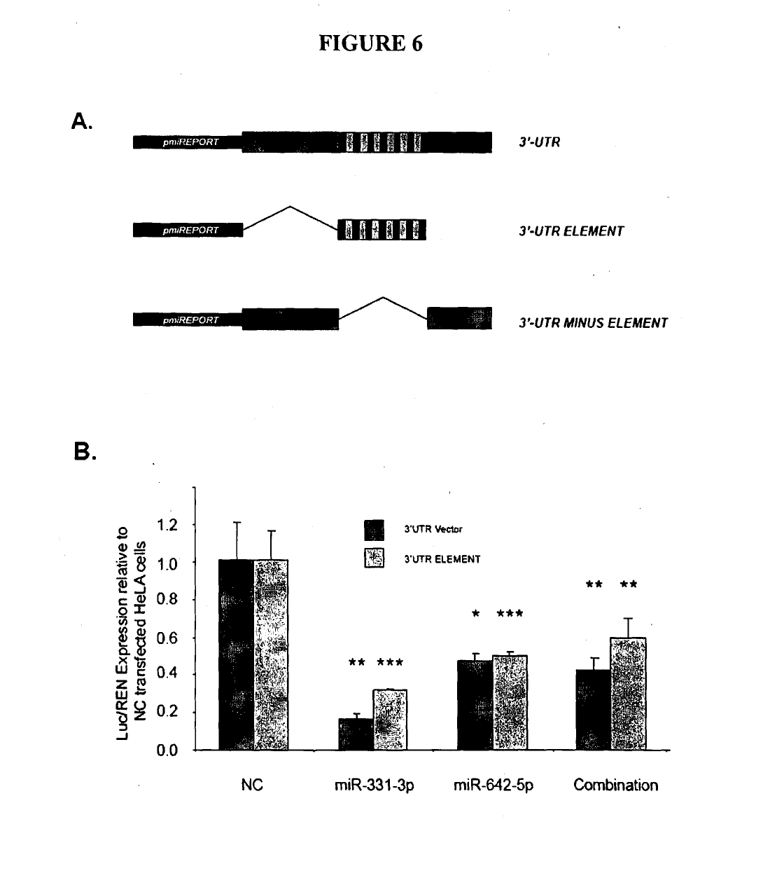

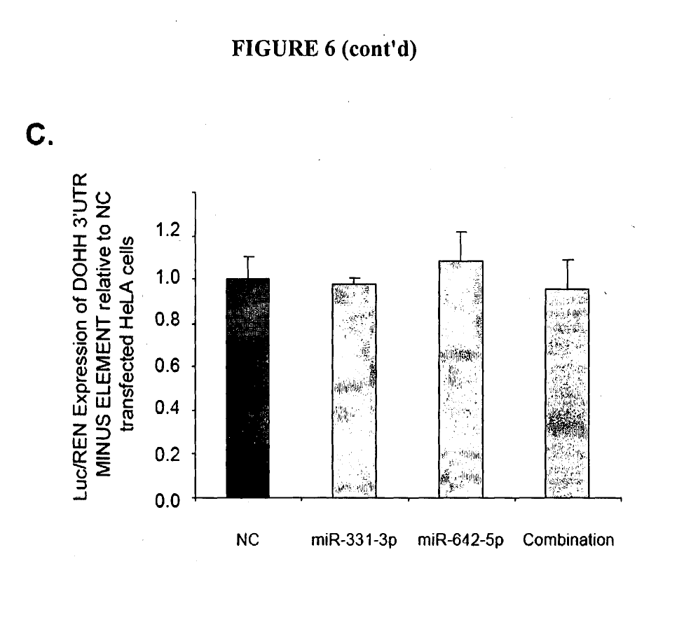

Briefly, three reporter gene constructs were prepared using a pmiR-REPORT vector (Ambion) as the backbone. The first construct was the construct described in Example 2, containing the DOHH 3′-UTR downstream of the firefly luciferase reporter gene (labelled “3′UTR”). The second construct contained a DOHH 3′-UTR in which the 182 base pair element had been deleted, downstream of the firefly luciferase reporter gene (labelled “3′UTR MINUS ELEMENT”). The third construct contained the 182 nucleotide element downstream of the firefly luciferase reporter gene (labelled “3′UTR ELEMENT”) (FIG. 6A).

In the first assay, performed essentially as described in Example 2 by transfection of HeLa cells, the firefly luciferase expression from the “3′UTR” and “3′UTR ELEMENT” constructs was assessed in the presence of hsa-miR-331-3p, hsa-miR-642-5p, a combination of the two or vehicle only (NC). As shown in FIG. 6B, both hsa-miR-331-3p and hsa-miR-642-5p, alone and in combination, could target “3′UTR” and “3′UTR ELEMENT” constructs, resulting in repression of firefly luciferase expression, indicating that the region of the 182 bp element was sufficient to facilitate repression by these miRNAs.

In the second assay performed by transfecting HeLa cells, the expression of the reporter gene from the “3′UTR MINUS ELEMENT” construct was assessed in the presence of hsa-miR-331-3p and/or hsa-miR-642-5p. As shown in FIG. 6C, no repression of the reporter gene by either hsa-miR-331-3p or hsa-miR-642-5p was observed, indicating that the DOHH 3′-UTR with the 182 nucleotide element removed had lost its ability to be regulated by hsa-miR-331-3p and/or hsa-miR-642-5p. These results demonstrate that hsa-miR-331-3p and hsa-miR-642-5p inhibit expression by targeting sites within the 182 nucleotide region corresponding to nucleotide positions 1632 to 1813 of the DOHH mRNA variant 1 set forth in SEQ ID NO:1, nucleotide positions 1343 to 1524 of the DOHH mRNA variant 2 set forth in SEQ ID NO:2, and nucleotide positions 271-452 of the 3′UTR of DOHH set forth in SEQ ID NO:3.

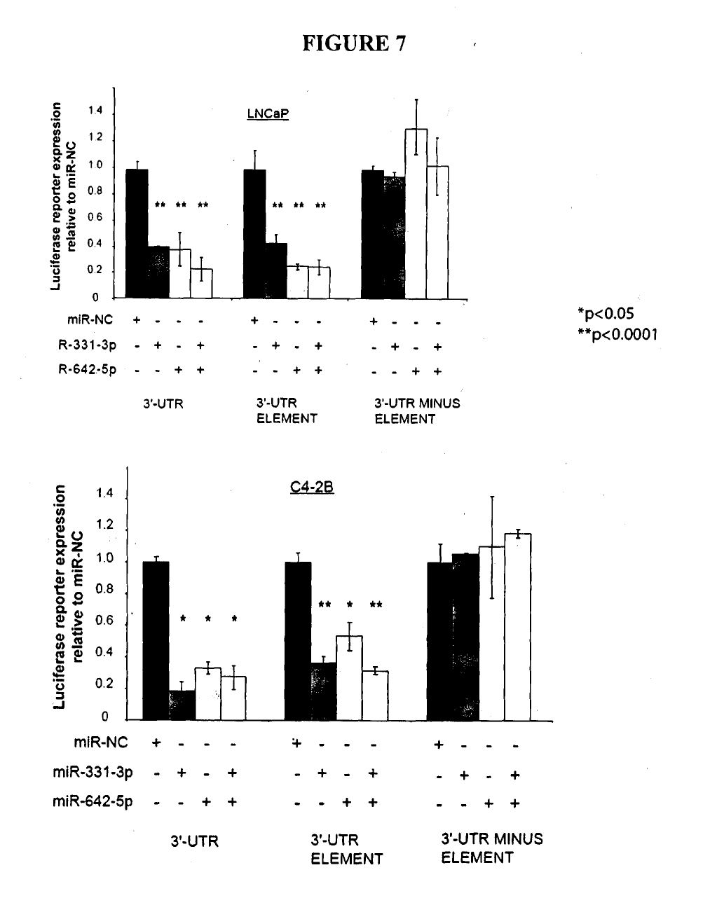

The effect of hsa-miR-331-3p and/or hsa-miR-642-5p on expression from the “3′UTR”, “3′UTR ELEMENT” and “3′UTR MINUS ELEMENT” construct in LNCaP and C4-2B prostate cancer cells was then assessed using the same transfection and assay techniques. Transfection with hsa-miR-331-3p and/or hsa-miR-642-5p significantly down-regulated reporter activity from the “3′UTR” and “3′UTR ELEMENT” constructs in both LNCaP and C4-2B cells, an effect that was not observed with the“3′UTR MINUS ELEMENT” construct lacking the 182 nucleotide element (FIG. 7).

These assays demonstrate that the 182 nucleotide element within the 3′-UTR of DOHH is a direct target of both hsa-miR-331-3p and hsa-miR-642-5p.

### Example 6

### Effect of Hsa-miR-331-3p and Hsa-miR-642-5p in DU145 Prostate Cancer Cells

The effect of transient transfection of DU145 prostate cells with hsa-miR-331-3p and hsa-miR-642-5p on DOHH expression and cell proliferation was investigated.

### A. Regulation of DOHH Expression in HeLa Cells

The effect of hsa-miR-331-3p and/or hsa-miR-642-5p on endogenous DOHH expression in DU145 prostate cancer cells was assessed by co-transfecting the cells with pre-miR-331-3p, pre-miR-642-5p, or pre-miR-NC essentially as described in Examples 2 and 3, and DOHH mRNA levels were assessed by qRT-PCR of DOHH mRNA. Parallel Western blotting experiments were performed to assess DOHH protein levels, as also described above.

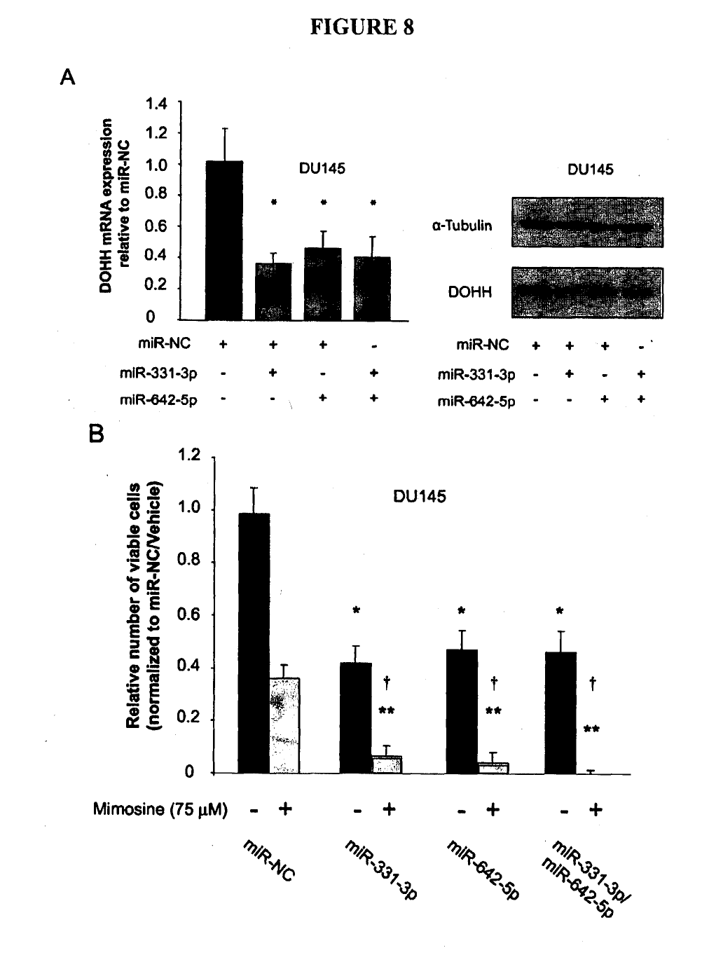

Transfection of DU145 cells with hsa-miR-331-3p and/or hsa-miR-642-5p significantly reduced expression of endogenous DOHH mRNA (FIG. 8A). The Western blotting experiments showed that transient transfection of miR-331-3p and/or miR-642-5p inhibited expression of DOHH protein (FIG. 8A).

DU145 cells were also co-transfected with the “3′UTR ELEMENT” construct (i.e. the luciferase reporter carrying the DOHH 3′-UTR 182 nucleotide element) and hsa-miR-331-3p and/or hsa-miR-642-5p. Reduced luciferase RNA levels were observed, consistent with a post-transcriptional effect of these miRNAs (data not shown).

Together, these results suggest that hsa-miR-331-3p and hsa-miR-642-5p regulate DOHH expression in the prostate by inducing decay of its mRNA, resulting in reduced levels of DOHH protein.

B. Effect of Hsa-miR-331-3p, Hsa-miR-642-5p and/or Mimosine on DU145 Cell Proliferation.

The functional significance of hsa-miR-331-3p and/or hsa-miR-642-5p overexpression in DU145 cells was then investigated. DU145 cells were transfected with pre-miR-331-3p, pre-miR-642-5p, or pre-miR-NC as described above. Cells were then treated with or without mimosine (75 μM) at 48 hours post transfection, and cell proliferation was assessed at 5 days post transfection with the CellTiter 96 Aqueous One Solution Cell Proliferation System (Promega) and a Fluostar Optima plate reader (BMG Scientific) essentially as described above in Example 3.

hsa-miR-331-3p and/or hsa-miR-642-5p significantly reduced DU145 cell proliferation when compared with miR-NC, an effect likely to be due in part to decreased DOHH expression and hence eIF5A activity. The combination of hsa-miR-331-3p and/or hsa-miR-642-5p, and mimosine produced a synergistic inhibition of DU145 cell growth (FIG. 8B). Using the Bliss model (Bliss (1939) Ann Appl. Biol 26:585-615), synergy occurs when the observed fractional inhibition for the combination of hsa-miR-331-3p and/or hsa-miR-642-5p and mimosine (defined as E_{observed}) exceeds the sum of the combined miRNA effect and the mimosine effect (defined as E_{Bliss}) (Table 3).

TABLE 3
Growth inhibition values for synergy calculations

| | | | Growth inhibition |
|---|---|---|---|
| | Growth inhibition | Growth inhibition | hsa-miR-331-3p/ |
| | hsa-miR-331- | hsa-miR-642-5p/ | hsa-miR-642-5p/ |
| E values | 3p/Mimosine | Mimosine | Mimosine |
| Eₐ (miRNA) | 0.572 | 0.523 | 0.532 |
| Eₐ (miRNA) | 0.632 | 0.632 | 0.632 |
| E_{Bliss} | 0.842 | 0.825 | 0.828 |
| E_{observed} | 0.933^{a} | 0.961^{a} | 1.014^{a} |

^{a}Synergy according to the Bliss Additivity model

Taken together, these results indicate that down-regulation of DOHH expression by hsa-miR-331-3p and/or hsa-miR-642-5p is associated with reduced DU145 prostate cancer cell proliferation and that this decrease in DOHH levels results in a synergistic growth inhibition when combined with the DOHH inhibitor mimosine.

### Example 7

### Expression of DOHH, Hsa-miR-331-3p and Hsa-miR-642-5p in Prostate Tumours and Matched Normal Adjacent Tissue

The expression of DOHH, hsa-miR-331-3p and hsa-miR-642-5p in a subset of prostate tumours and matched normal adjacent tissue was assessed to determine whether there was any correlation. Ten prostate cancer samples were obtained with matching normal adjacent tumour (NAT). The tissue samples were homogenised before RNA extraction by pulsing samples at 2×45 second pulses using 2.8 mm ceramic beads in a Precellys® 24 Homogeniser (Bertin Technologies). The total RNA was then extracted using Qiazol reagent (Qiagen) as per the manufacturer's instructions.

DOHH mRNA expression was determined by qRT-PCR as described in Example 3 above. Levels of hsa-miR-331-3p and hsa-miR-642-5p were assessed using the Taqman® miRNA Assay system (Applied Biosystems). Briefly, 10 ng of total RNA was used and reverse transcription was performed according to manufacturers instruction using a specific Taqman® RT Primer for hsa-miR-331, hsa-miR-642-5p and RNU6B (Applied Biosystems; Assay ID: 000545; Assay ID: 001592; Assay ID: 001093). Detection of mature miRNAs was performed using specific Taqman® miRNA PCR detection probes for each miRNA or small RNA. Expression of hsa-miR-331-3p and hsa-miR-642-5p mature miRNA relative to RNU6B small RNA was determined using the 2^{-ΔCt} method and statistical analysis of qRT PCR data was performed using GenEx software (MultiD).

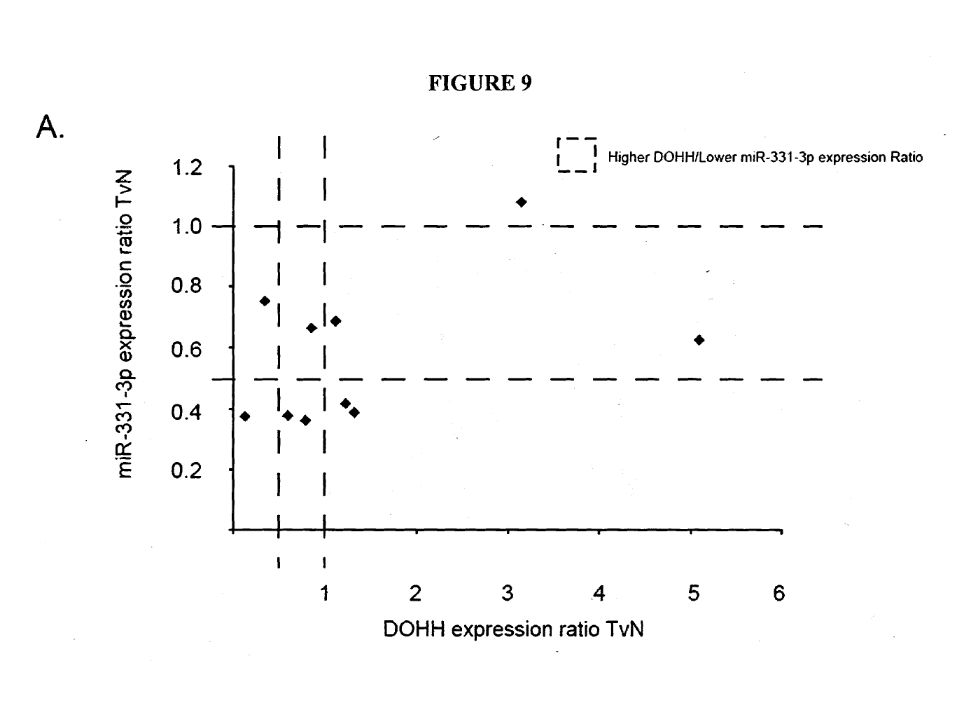

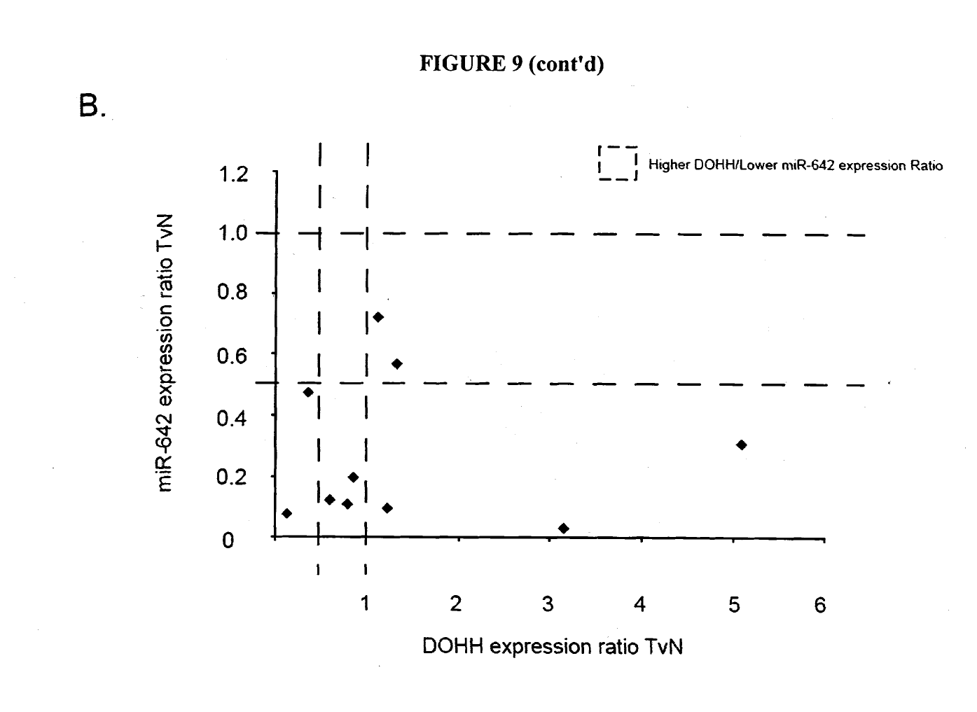

Individual expressions for DOHH, hsa-miR-331-3p and hsa-miR-642-5p were detected and normalised and scatter plots were used to represent the data of miRNA expression vs DOHH expression as a Tumour vs NAT ratio (FIG. 9A, B).

To stratify the cohort, a threshold of a ratio of 1 was set, which was the point where DOHH and miRNA expression was equal. As seen in FIG. 9, data points within the shaded area are representative of a lower than ratio 1 miRNA expression with a higher than a ratio of 1 DOHH expression. This is representative of samples that have an inverse relationship between miRNA expression and DOHH expression, i.e. samples that have a low x-axis ratio and a higher y-axis ratio and fit the hypothesis that a down regulation of the miRNA in tumours leads to an up regulation of the target mRNA. Samples outside the shaded area are representative both low DOHH expression with low miRNA expression and high miRNA expression with high DOHH expression. Of particular note, both scatter plots for hsa-miR-331-3p and hsa-miR-642-5p contain the majority of data points the shaded area, with 6 pairs found for hsa-miR-331-3p (FIG. 9A) and 8 pairs for hsa-miR-642-5p (FIG. 9B). These results indicate that in this Tumour vs NAT cohort, tumour tissues exhibited reduced miRNA expression, which correlated with an up regulation of the specific target: DOHH.

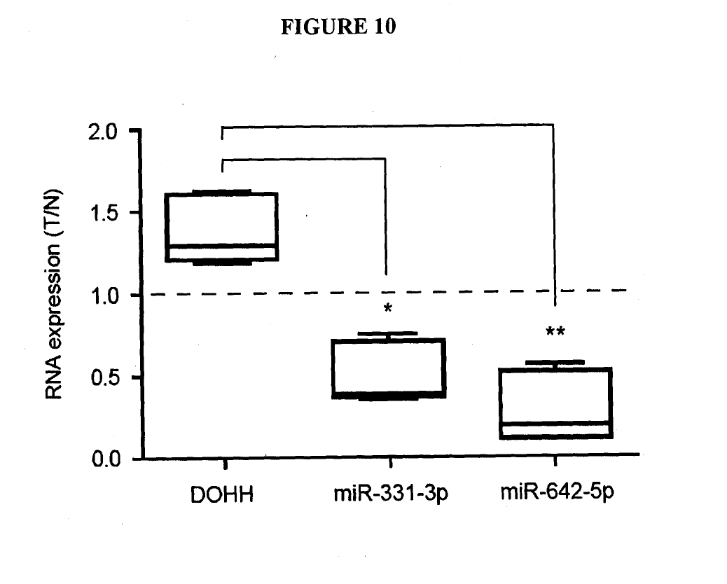

It was observed that five of nine (56%) tumor samples had elevated DOHH mRNA expression. Using TaqMan miRNA RT-qPCR, it was observed that expression of hsa-miR-331-3p and hsa-miR-642-5p was significantly reduced in each instance versus the tumor sample's matched NAT (FIG. 10). hsa-miR-331-3p and hsa-miR-642-5p expression was also reduced in the subset of four tumor samples lacking DOHH overexpression (data not shown).

These findings suggest that loss of miR-331-3p and miR-642-5p may facilitate the elevated expression of DOHH and subsequent cellular growth, in part via eIF5A activation, in prostate cancer.

What is claimed is:

1. A method of inhibiting expression of deoxyhypusine hydroxylase monooxygenase (DOHH) in a cell, comprising contacting the cell with a miRNA or a nucleic acid molecule encoding the miRNA, wherein the miRNA binds to the 3′UTR of the DOHH mRNA and wherein binding results in a reduction in DOHH expression.

2-3. (canceled)

4. The method of claim 1, wherein the cell is selected from among a cancer cell, a HIV-infected cell and an islet β cell.

5. The method of claim 1, wherein the cell is in a subject with a disease associated with abnormal cellular proliferation, and the method comprises administering to the subject the miRNA or nucleic acid molecule encoding the miRNA to treat the disease.

6. The method of claim 5, wherein the disease associated with abnormal cellular proliferation is a cancer.

7. The method of claim 6, wherein the cancer is selected from among the group consisting of biliary tract cancer; bladder cancer; breast cancer; brain cancer; glioblastoma; medulloblastoma; cervical cancer; choriocarcinoma; colon cancer; colorectal carcinoma; endometrial cancer; esophageal cancer; gastric cancer; head and neck cancer; hematological neoplasms, acute lymphocytic and myelogenous leukemia; multiple myeloma; AIDS-associated leukemia and adult T-cell leukemia lymphoma; intraepithelial neoplasms, Bowen's disease; Paget's disease; liver cancer; lung cancer; small cell lung cancer; non-small cell lung cancer; lymphoma; Hodgkin's disease; lymphocytic lymphoma; neuroblastoma; oral cancer; squamous cell carcinoma; osteosarcomas; ovarian cancer; pancreatic cancer; prostate cancer; rectal cancer; sarcoma; leiomyosarcoma; rhabdomyosarcoma; liposarcoma; fibrosarcoma; synovial sarcoma; osteosarcoma; skin cancer; melanoma; Kaposi's sarcoma; basocellular cancer; squamous cell cancer; testicular cancer; thyroid cancer; renal cancer; adenocarcinoma and Wilms tumor.

8-12. (canceled)

13. The method of claim 1, further comprising contacting the cell with the anti-proliferative agent, wherein contacting the cell with the miRNA or the nucleic acid molecule encoding the miRNA and the anti-proliferative agent enhances the effect of the anti-proliferative agent compared to the effect of the anti-proliferative agent alone.

14. The method of claim 1, wherein the miRNA binds to one or more target sites between nucleotides 270 to 470, 271 to 452, 280 to 470, 280 to 460, 280 to 452, or 286 to 452 of the human DOHH mRNA 3′UTR set forth in SEQ ID NO:3 or corresponding nucleotides in a DOHH mRNA 3′-UTR of another species.

15. The method of claim 14, wherein that target site is selected from among the target sites set forth in nucleotides 286-292, 308-315, 331-338, 354-361, 377-384, 423-430 and 446-452 of the DOHH 3′-UTR set forth in SEQ ID NO: 3 or corresponding nucleotides in a DOHH mRNA 3′-UTR of another species.

16. The method of claim 14, wherein that target site is selected from among the target sites set forth in nucleotides 300-306, 323-329, 346-352, 369-375, 392-398 and 438-444 of the human DOHH mRNA 3′UTR set forth in SEQ ID NO:3 or corresponding nucleotides in a DOHH mRNA 3′-UTR of another species.

17. The method of claim 1, wherein the miRNA comprises a sequence of nucleotides set forth in SEQ ID NO:8 and wherein the nucleotides set forth in SEQ ID NO:8 mediate binding of the miRNA to the 3′-UTR of the DOHH mRNA.

18. The method of claim 1, wherein the miRNA comprises a sequence of nucleotides set forth in SEQ ID NO:9 and wherein the nucleotides set forth in SEQ ID NO:9 mediate binding of the miRNA to the 3′-UTR of the DOHH mRNA.

19. The method of claim 1, wherein the miRNA is hsa-miR-331-3p and comprises a sequence set forth in SEQ ID NO:4 or is a variant thereof that has at least or about 80% sequence identity to the sequence set forth in SEQ ID NO:4.

20. The method of claim 1, wherein the miRNA is hsa-miR-642-5p and comprises a sequence set forth in SEQ ID NO:5 or is a variant thereof that has at least or about 80% sequence identity to the sequence set forth in SEQ ID NO: 5.

21. The method of claim 1, wherein the nucleic acid encoding the miRNA comprises or encodes the hsa-miR-331 precursor comprising a sequence set forth in SEQ ID NO:6 or a variant thereof that has at least or about 80% sequence identity to the sequence set forth in SEQ ID NO:6.

22. The method of claim 1, wherein the nucleic acid encoding the miRNA comprises or encodes the hsa-miR-642-5p precursor comprising a sequence set forth in SEQ ID NO:7 or a variant thereof that has at least or about 80% sequence identity to the sequence set forth in SEQ ID NO:7.

23. (canceled)

24. The method of claim 1, wherein two or more miRNAs or nucleic acid molecules encoding two or more miRNAs are contacted with the cell.

25. (canceled)

26. The method of claim 1, further comprising contacting the cell with an additional therapeutic agent.

27. The method of claim 26, wherein the therapeutic agent is selected from among an anti-cancer agent, anti-viral agent, anti-diabetic agent, immunomodulatory agent and an anti-proliferative agent.

28. (canceled)

29. The method of claim 26, wherein the therapeutic agent inhibits DOHH or DHS activity.

30. (canceled)

31. The method of claim 26, wherein the therapeutic agent inhibits DOHH activity and is selected from among the group consisting of mimosine, deferiprone and ciclopirox.

32-33. (canceled)

34. A composition, comprising hsa-miR-331-3p comprising a sequence set forth in SEQ ID NO:4 or a variant thereof that has at least or about 80% sequence identity to the sequence set forth in SEQ ID NO:4; and hsa-miR-642-5p comprising a sequence set forth in SEQ ID NO:5 or a variant thereof that has at least or about 80% sequence identity to the sequence set forth in SEQ ID NO:5.

35. A method of detecting cancer cells in a subject, comprising measuring the level of hsa-miR-642-5p in a subject; and
comparing the level to a reference level of hsa-miR-642-5p; wherein
cancer cells are detected if the level of hsa-miR-642-5p in the subject is decreased compared to the reference level.

36-44. (canceled)

45. The method of claim 35, further comprising measuring the level of deoxyhypusine hydroxylase monooxygenase (DOHH) mRNA or protein, wherein the ratio of DOHH mRNA or protein to hsa-miR-642-5p is determined and compared to a reference ratio of DOHH mRNA or protein to hsa-miR-642-5p, and wherein cancer is detected if the ratio of DOHH mRNA or protein to hsa-miR-642-5p in the subject is increased as compared to the reference ratio.

46. (canceled)

47. The method of claim 35, wherein the cancer is prostate cancer.

48-50. (canceled)


SEQUENCE LISTING (18 sequences)

SEQ ID NO: 1 - RNA, 2050 nt, Homo sapiens
agccagcccaggcagggcgcugaaucggcacaaaccagucucacuucuuguaccgcaguggggagguuggagagaauuuuuucccaugaaagggcauacguugcccguccccuuccccgaagaacgcacuaggaauacuguuccugcguuugcaugagcuguggcaccagcaccgacucagccuuccgggccucccucaugcacggagccucucacuaggucucacccgcggcugcguuggugccguccggcacggucacgugggcgagaaaauggcggcccccagaccgcacugcgggcgucgcggcaggugaagcggugguggcagaggcgccugcgguuacuggccgggccgacggguccugagucuccagagcucgguucuuacauccccgcguccccagguuuagccugagcaggguuguggaaggccgggacccauugacagcacgauggugacggagcaggagguggaugccaucgggcagacgcugguggaccccaagcagccccugcggcccgcuuccgggcgcuguucacgcugcgugggcucggcggcccaggcgccauugcauggaucagccaggccuucgaugacgauuccgcccugcucaagcacgagcuggccuacugccugggccagaugcaggaugcccgcgccauccccaugcugguggacgugcugcaagacacccgucaggagcccauggugcgccaugaggcaggggaggcccugggggccaucggggacccggaaguucuggagauccugaagcaguauuccucggaccccgucaucgagguggccgagaccugccagcuggccgugcgcaggcuggaguggcugcagcagcacggcggggagccggcggcgggacccuaccucuccguggacccugccccgccggcugaggagcgugacguggggcgccugcgggaggcgcugcuggaugagucccggccgcucuucgagcgauaccgcgccauguucgcccugcgcaacgcgggaggcgaggaggccgcccuggcgcuggccgagggucugcacugugggagcgcccucuuccgccacgaggucggcuacguccugggacagcugcagcacgaggcggcggugccccagcuggcggccgcccuggcccgaugcaccgagaaccccauggugcggcacgagugcgcggaggcccugggcgccauugcccggcccgccugccuggccgcgcugcaggcucacgcggacgacccagagcgcguggugcgugagagcugcgagguggcucuggacauguaugagcacgagaccgggcgggccuuccaguacgcggacggccuggagcagcugcgcggggcccccuccuagggccccacccucacccggagcucccggaggacucuugagggccgcuccucccccgcagagcuuuggcgucuaaaccggguguguguaaaucggugucaucgcuugugucuugcugggcgcaugguugcugucccccuccuccgucugggaccgaggagcugccguugugcugugacucccugaguccccugacugcuccucggggccuggcgcagggcuguagcguggaaguuuccaggcuuggggugggacucugcgggaggcucugggaugcccggcucaggacaggggaggauugcgagggaagccaggggaggaucgcgagggaagccaggggaggaucgcgagggaagccaggggaggaucgcgagggaagccaggggaggaucgcgagggaagccaagggaggaucgcgaggggagccaggggaggaucgcgagggaagccaggggcagagcuggggcucugggaagaggcugacguuaugguggcuucagcuucacuaggaaugggacacagggucugggggccucugacucccccaccccgaggccuggguagggacagggugguggucccugagugggucagguagggcacaggggccagggagggacaagcagaccucagagcgcugcccagauggaauauuaaauuauuuuugccaagcaaaaaaaaaaaaaaaaaaaa

SEQ ID NO: 2 - RNA, 1761 nt, Homo sapiens
ggugguggcagaggcgccugcgguuacuggccgggccgacggguccugagucuccagagcucggugaggcgucccggaggcggcgaucacgguucuuacauccccgcguccccagguuuagccugagcaggguuguggaaggccgggacccauugacagcacgauggugacggagcaggagguggaugccaucgggcagacgcugguggaccccaagcagccccugcaggcccgcuuccgggcgcuguucacgcugcgugggcucggcggcccaggcgccauugcauggaucagccaggccuucgaugacgauuccgcccugcucaagcacgagcuggccuacugccugggccagaugcaggaugcccgcgccauccccaugcugguggacgugcugcaagacacccgucaggagcccauggugcgccaugaggcaggggaggcccugggggccaucggggacccggaaguucuggagauccugaagcaguauuccucggaccccgucaucgagguggccgagaccugccagcuggccgugcgcaggcuggaguggcugcagcagcacggcggggagccggcggcgggacccuaccucuccguggacccugccccgccggcugaggagcgugacguggggcgccugcgggaggcgcugcuggaugagucccggccgcucuucgagcgauaccgcgccauguucgcccugcgcaacgcgggaggcgaggaggccgcccuggcgcuggccgagggucugcacugugggagcgcccucuuccgccacgaggucggcuacguccugggacagcugcagcacgaggcggcggugccccagcuggcggccgcccuggcccgaugcaccgagaaccccauggugcggcacgagugcgcggaggcccugggcgccauugcccggcccgccugccuggccgcgcugcaggcucacgcggacgacccagagcgcguggugcgugagagcugcgagguggcucuggacauguaugagcacgagaccgggcgggccuuccaguacgcggacggccuggagcagcugcgcggggcccccuccuagggccccacccucacccggagcucccggaggacucuugagggccgcuccucccccgcagagcuuuggcgucuaaaccggguguguguaaaucggugucaucgcuugugucuugcugggcgcaugguugcugucccccuccuccgucugggaccgaggagcugccguugugcugugacucccugaguccccugacugcuccucggggccuggcgcagggcuguagcguggaaguuuccaggcuuggggugggacucugcgggaggcucugggaugcccggcucaggacaggggaggauugcgagggaagccaggggaggaucgcgagggaagccaggggaggaucgcgagggaagccaggggaggaucgcgagggaagccaggggaggaucgcgagggaagccaagggaggaucgcgaggggagccaggggaggaucgcgagggaagccaggggcagagcuggggcucugggaagaggcugacguuaugguggcuucagcuucacuaggaaugggacacagggucugggggccucugacucccccaccccgaggccuggguagggacagggugguggucccugagugggucagguagggcacaggggccagggagggacaagcagaccucagagcgcugcccagauggaauauuaaauuauuuuugccaagcaaaaaaaaaaaaaaaaaaa

SEQ ID NO: 3 - RNA, 689 nt, Homo sapiens
ggccccacccucacccggagcucccggaggacucuugagggccgcuccucccccgcagagcuuuggcgucuaaaccggguguguguaaaucggugucaucgcuugugucuugcugggcgcaugguugcugucccccuccuccgucugggaccgaggagcugccguugugcugugacucccugaguccccugacugcuccucggggccuggcgcagggcuguagcguggaaguuuccaggcuuggggugggacucugcgggaggcucugggaugcccggcucaggacaggggaggauugcgagggaagccaggggaggaucgcgagggaagccaggggaggaucgcgagggaagccaggggaggaucgcgagggaagccaggggaggaucgcgagggaagccaagggaggaucgcgaggggagccaggggaggaucgcgagggaagccaggggcagagcuggggcucugggaagaggcugacguuaugguggcuucagcuucacuaggaaugggacacagggucugggggccucugacucccccaccccgaggccuggguagggacagggugguggucccugagugggucagguagggcacaggggccagggagggacaagcagaccucagagcgcugcccagauggaauauuaaauuauuuuugccaagcaaaaaaaaaaaaaaaaaaa

SEQ ID NO: 4 - RNA, 21 nt, Homo sapiens
gccccugggccuauccuagaa

SEQ ID NO: 5 - RNA, 22 nt, Homo sapiens
gucccucuccaaaugugucuug

SEQ ID NO: 6 - RNA, 94 nt, Homo sapiens
gaguuugguuuuguuuggguuuguucuagguauggucccagggaucccagaucaaaccaggccccugggccuauccuagaaccaaccuaagcuc

SEQ ID NO: 7 - RNA, 97 nt, Homo sapiens
aucugaguugggagggucccucuccaaaugugucuuggggugggggaucaagacacauuuggagagggaaccucccaacucggccucugccaucauu

SEQ ID NO: 8 - RNA, 7 nt, Homo sapiens
ccccugg

SEQ ID NO: 9 - RNA, 6 nt, Homo sapiens
ucccuc

SEQ ID NO: 10 - DNA, 20 bp, Artificial Sequence
  DOHH-F91
ggccgaggggtcctgagtct

SEQ ID NO: 11 - DNA, 20 bp, Artificial Sequence
  DOHH R91
tgggtcccggccttccacaa

SEQ ID NO: 12 - DNA, 20 bp, Artificial Sequence
  DOHH F191
cagggcgctgaatcggcaca

SEQ ID NO: 13 - DNA, 20 bp, Artificial Sequence
  DOHH R191
tgcatgagggaggcccggaa

SEQ ID NO: 14 - DNA, 19 bp, Artificial Sequence
  GAPDH-F
atggggaaggtgaaggtcg

SEQ ID NO: 15 - DNA, 22 bp, Artificial Sequence
  GAPDH-R
ggggtcattgatggcaacaata

SEQ ID NO: 16 - DNA, 20 bp, Artificial Sequence
  B-actin-F
gccaacacagtgctgtctgg

SEQ ID NO: 17 - DNA, 20 bp, Artificial Sequence
  B-actin-R
tactcctgcttgctgatcca

SEQ ID NO: 18 - DNA, 527 bp, Macaca mulatta
ggccccgccctcacctggggctccctgaggattcttgagggccgctcctcccccgcagggctttggcgtctaaactgagtgtgtgtgtgaatcggtgtcatcgcttgtgtcttgctgggtgcacggttgctgtccttctctgggacccaggagctgcctttgtgctgtgactccctgagtccactggcagctccccggggcctagcgcagggctgtagggtggaagtttccaggttcaggggtgggactctgcgggaggctctgggatgcccggctcaggacaggggaggattgccagggaagccaggggcagagctggagctcttggaagaggctggcattatggtggcttcagtttcactaggaatgaggcccagggtctgggggcctctgactcccccacccccaggcctgggcagggatggggtggtggtccctgagtgggtcaggtggggcacagcggccagggagggacaagcagaccccacagcgctgcccagatggaatattaaattatttttgccaaa